(19) United States
(12) Patent Application Publication
(10) Pub. No.: US 2008/0119483 A1
(43) Pub. Date: May 22, 2008

(54) COMPOSITIONS AND METHODS FOR INHIBITING PROTOZOAN GROWTH

(75) Inventors: Stephen M. Beverley, Clayton, MO (US); Kai Zhang, St. Louis, MO (US)

Correspondence Address: POLSINELLI SHALTON FLANIGAN SUELTHAUS PC, 100 SOUTH FOURTH STREET, SUITE 100, SAINT LOUIS, MO, 63102-1825, US

(73) Assignee: Washington University in St. Louis, St. Louis, MO (US)

(21) Appl. No.: 11/840,741

(22) Filed: Aug. 17, 2007

Related U.S. Application Data
(60) Provisional application No. 60/822,808, filed on Aug. 18, 2006.

(51) Int. Cl. A61K 31/496; A01P 15/00; A61K 45/00; A61P 31/00

(52) U.S. Cl. 514/254.07; 514/789

(57) ABSTRACT

The present invention provides compositions and methods for inhibiting protozoan growth comprising a synergistic combination of lipid synthesis inhibitors. In addition, the invention provides compositions and methods that are useful for the treatment of protozoan infections and the identification of potential new drugs for the treatment of protozoan infections.

### RELATED APPLICATIONS

This application claims priority to U.S. Provisional Application No. 60/822,808, filed Aug. 18, 2006, which is commonly owned and incorporated herein in its entirety by reference.

### FIELD OF THE INVENTION

The present invention relates to compositions and methods for inhibiting protozoan growth. Compositions and methods of the invention also may be used to treat protozoan infections and/or disorders relating to a protozoan infection.

### BACKGROUND OF THE INVENTION

Protozoa are unicellular, eukaryotic microorganisms that can infect mammals, insects, and birds. Some clinically important representatives include Giardia lamblia, Plasmodium spp., Toxoplasma, Trichomonas vaginalis, Leishmania spp., and Trypanosoma spp. G. lamblia is a waterborne intestinal parasite that causes diarrhea and other intestinal symptoms. The most commonly used drugs used to treat giardiasis are metronidazole and other members of the 5-nitroimidazoles. Unfortunately, metronidazole is mutagenic according to the Ames test (Vogd et al., Mutation Research, vol. 26, 483 490 (1974)) and has various undesirable toxic side effects. In addition, the development of resistance to these drugs in Giardia and other protozoan parasites such as Entamoeba histolytica and Trichomonas vaginalis also limits their effectiveness.

Leishmaniasis is a life-threatening disease caused by Leishmania spp. that is a major health problem worldwide. An estimated 10-15 million people are infected, and 400,000 new cases occur each year. Currently no vaccine is available against Leishmania and the generic, antimony-based drug treatments are plagued with low efficacy, high toxicity and widespread resistance [Croft and Coombs, 2003]. To control these dangerous pathogens, new drugs and novel therapeutic strategies are in dire need.

Similarly, other protozoa cause other serious diseases in humans and animals. For example, Trypanosoma spp. cause life-threatening diseases in humans, including African sleeping sickness and Chagas disease, as well as a number of important diseases in domestic animals. Leishmania and Trypanosoma are closely-related genera, representing the major pathogens in the kinetoplastid group of protozoa.

The intestinal parasite Entamoeba histolytica causes amoebic dysentery and extraintestinal abscesses of organs such as the liver and lung. The most commonly used drug for treating E. histolytica infection is metronidazole. Other free-living amoeba, which occasionally cause infections in humans, include Acanthamoeba and Naegleria spp.; these infections are typically difficult to treat.

Additional important protozoans include the malaria parasite Plasmodium spp.; the water-born pathogen Cryptosporidium spp. important in human health; Toxoplasma gondii; and several protozoans of veterinary importance such as Sarcocystis spp.; Theileria spp.; Babesia spp.; and Eimeria spp. (causing coccidiosis in fowl and domestic animals). Cryptosporidium parvum is a common cause of intestinal infection leading to self-limited diarrhea, but in the immuno-compromised individual C. parvum infection is chronic and life-threatening. There is currently no effective treatment for cryptosporidiosis.

Toxoplasmosis is among the most common parasitic diseases of man. Serosurveys suggest prevalence rates as high as 70-90% in many areas of both the developing and developed world. Between 10-45% of Americans become infected at some point in their lives. Toxoplasma gondii is the causative agent in toxoplasmosis. In contrast to the mild clinical symptoms of infection seen in a healthy individual with an intact immune system, subjects with weakened, or otherwise compromised, immune systems can have serious clinical effects from toxoplasma infection. Toxoplasma gondii is also pathogenic to animals, particularly sheep, in which it causes abortion, stillbirth, and fetal mummification. In addition, Toxoplasma gondii causes encephalitis, a dangerous life-threatening disease in both man and domestic animals.

The World Health Organization (WHO) estimates that 300-500 million people are infected by malaria each year and that more than 2 million people, mostly women and children under the age of five, die from malaria annually. Plasmodium falciparum causes a severe form of human malaria and is responsible for nearly all malaria-specific mortality. Resistance of Plasmodium to anti-malarial drugs is an increasingly serious problem in fighting the disease.

Ticks transmit babesiosis, and although this is primarily a disease of animals, humans are also infected with this parasite. There are over 100 species of Babesia, with Babesia microti and Babesia divergens the two most likely to cause human infection. Babesia microti is the organism responsible for a growing number of cases of infection especially in the northeast United States. Babesiosis is not only transmitted via tick bites, it can also be transmitted via blood transfusions, with documented cases of infection via this method.

Sarcocystis parasites may be ingested by humans in undercooked meat, and once in the body, they may cause intestinal infections. More commonly, the sporocysts are ingested via fecal contamination, after which the sporocysts may result in cyst formation in striated muscle and cardiac muscle in the host. Additionally, Sarcocystis neurona is the responsible agent for equine protozoal myeloencephalitis, a debilitating disease caused by protozoal infection of the central nervous system.

Cryptosporidosis is a common infection in subjects with compromised immune systems. Like sarcosporidiosis, oocysts of the parasites are ingested via fecal contamination. The oocysts release sporozoites that infect epithelial cells of the intestinal tract resulting in severe and at times life-threatening diarrheal disease.

Theileria infection is transmitted by ticks and results in disorders such as East Coast Fever and Mediterranean Coast Fever. Following infection, the protozoans are located in the host's red blood cells and clinical symptoms include fever, weight loss, enlarged lymph nodes and spleen, mild anemia, and possible pulmonary edema. Theileria infections can be fatal to cattle and have a significant impact on the economy of sub-Saharan Africa.

There are numerous species of Eimeria, and an oral/fecal route of transmission results in intestinal infection in cows, sheep, goats, pigs, ducks, chickens, turkeys, and rabbits, with the domestic chicken host to at least seven different species of Eimeria. Due to its widespread nature and its effects on the host animal, which may result in sub-optimal weight gain and reduced economic value, Eimeria is an economically important disease in the modern poultry production.

Additionally, Encephalitozoon species (including E. intestinalis, E. cuniculi, and E. hellem and Enterocytozoon bieneusi) can cause disease in mammals, fish and invertebrates.

As mentioned above, effective therapies against Leishmania spp. And other protozoa are lacking. Ergosterol synthesis pathway has long been a favorable target for anti-fungal chemotherapy [Georgopapadakou, 1998; Georgopapadakou and Walsh, 1996]. Like fungi, Leishmania parasites synthesize high level of ergosterol de novo [Holz, et al., 1985]. Several anti-fungal agents targeting ergosterol synthesis have been used to treat Leishmania infections, including terbinafine (inhibitor of squalene epoxidase, EC 1.14.99.7; [Kirkpatrick et al., 2005; Perez, 1999]), itraconazole, ketoconazole and fluconazole (inhibitors of the cytochrome P450-dependent lanosterol 14-alpha-demethylase or C14DM, EC 1.14.13.70, [Vanden Bossche, et al., 1998; Martin, 1999; Bailey, et al., 1990]). It is believed that these inhibitors cause depletion of endogenous ergosterols and eventually lead to cell death in Leishmania parasites, similar to their mode of action in fungi [Hart, et al., 1989; Goad, et al., 1985]. Besides ergosterol, plasma membranes of Leishmania parasites also contain abundant amounts (more than 108 molecules per cell) of ether phospholipids (EPLs) and sphingolipids (SLs, mostly in the form of inositol phosphorylceramide or IPC) [Kaneshiro, et al., 1986; van der Rest, et al., 1995; Zufferey, et al., 2003]. Both ergosterols and IPC are enriched in the detergent resistant membrane fractions (DRMs) and thought to promote the formation of organized membrane microdomains known as lipid rafts [Brown, 2000; London, et al., 2000]. While less extensively studied, EPLs are also preferentially associated with lipid rafts and may promote the formation of rafts-like domains [Mattjus, and Slotte, 1996; Ohvo-Rekila, et al., 2002; Pike, L. J., et al., 2002].

It will be appreciated that there is an urgent need for new therapeutic agents to combat protozoan infections, which are sufficiently effective, do not have harmful side effects, and are not difficult or expensive to administer. Preferably, the anti-protozoal compounds are active against a broad spectrum of protozoa, while remaining non-toxic to humans, other mammalian cells, as well as avian cells.

### SUMMARY OF THE INVENTION

The present invention provides compositions for inhibiting protozoan growth. Advantageously, compositions of the invention inhibit protozoan growth with only minimal or no disruption or harm to the host.

Typically, the composition comprises a combination of two lipid synthesis inhibitors, preferably from different classes or groups of inhibitors. The combination of the two lipid synthesis inhibitors acts synergistically to provide greater inhibition of protozoan growth than would be expected by either inhibitor alone or additively together. For example, the EC50 of a composition of the invention would be at least 10-fold lower than the individual EC50s for either of the lipid synthesis inhibitors. More preferably, the EC50 of a composition of the invention would be 20-fold, 50-fold, 100-fold, or even 1000-fold or more less than the individual EC50s for either of the lipid synthesis inhibitors. Herein, the “EC50” value is the concentration of agent needed to inhibit growth of the protozoan by 50% compared to a control with no agent. The lower the EC50 value, the more potent the agent is at inhibiting protozoan growth.

Stated another way, the ratio of the EC50 of the first lipid synthesis inhibitor alone (EC50₁) to the EC50 of a composition of the invention (EC50_{c}) is ten or greater, and the ratio of the EC50 of the second lipid synthesis inhibitor alone (EC50₂) to the EC50_{c} also is ten or greater. Thus, the ratio of the EC50 for an individual inhibitor to the EC50 of both inhibitor combined provides a measure of the additive or synergistic effects of both inhibitors. If the ratio is equal to 1, there is no additive or synergistic effect. If the ratio is less than 1, the combination of inhibitors is antagonistic to inhibiting protozoan growth. In contrast, if the ratio is greater than 1, the combination possesses additive or synergistic activity. In preferred embodiments of the present invention, the ratio of an individual inhibitor to the combination of inhibitors is ten or greater, which is indicative of a synergistic effect. An additive effect would be expected to produce a ratio of two or possibly three. More preferably, the ratio of the EC50 of an individual inhibitor to the EC50 of a combination of inhibitors will be at least 20, 50, 100, or even 1000 or more.

The present invention provides compositions for inhibiting protozoan growth comprising a first lipid synthesis inhibitor and a second lipid synthesis inhibitor, wherein the ratio of the EC50 of the first lipid synthesis inhibitor alone to the EC50 of the composition is at least ten, twenty, fifty or even one hundred and the ratio of the EC50 of the second lipid synthesis inhibitor alone to the EC50 of the composition is at least ten, twenty, fifty or even one hundred.

In certain embodiments, the first lipid synthesis inhibitor is an ergosterol synthesis inhibitor, a sphingolipid synthesis inhibitor, or an ether phospholipid synthesis inhibitor, such as an alkyl-dihydroxyacetonephosphate transferase inhibitor. An exemplary ergosterol synthesis inhibitor is a lanosterol 14α-demethylase inhibitor such as itraconazole or ketaconazole. Exemplary sphingolipid synthesis inhibitors are a serine palmitoyltransferase inhibitor, ceramide synthase inhibitor, an inositol phosphorlyceramide synthase inhibitor, glycosyl ceramide synthase inhibitor, or a sphingolipid salvage inhibitor.

Preferably, the second lipid synthesis inhibitor is selected from a different class of lipid synthesis inhibitors than the first lipid synthesis inhibitor. For example, the first lipid synthesis inhibitor may be an ergosterol synthesis inhibitor, and the second lipid synthesis inhibitor may be a sphingolipid synthesis inhibitor. Alternatively, the first lipid synthesis inhibitor is an ergosterol synthesis inhibitor, and the second lipid synthesis inhibitor is an ether phospholipid synthesis inhibitor. Or in another alternative, the first lipid synthesis inhibitor is a sphingolipid synthesis inhibitor, and the second lipid synthesis inhibitor is an ether phospholipid synthesis inhibitor.

The invention also provides methods of inhibiting protozoan growth that comprise contacting the protozoan with an effective amount of a composition comprising a first lipid synthesis inhibitor and a second lipid synthesis inhibitor, wherein the ratio of the EC50 of the first lipid synthesis inhibitor alone to the EC50 of the composition is at least ten, twenty, fifty or even one hundred and the ratio of the EC50 of the second lipid synthesis inhibitor alone to the EC50 of the composition is at least ten, twenty, fifty or even one hundred.

Exemplary protozoans whose growth may be inhibited by the compositions and methods of the invention include Giardia, Trichomonas, Leishmania, Trypansosoma, Entamoeba, Plasmodium, Cryptosporidium, Toxoplasma, Sarcocystis, Theileria, Babesia, and Eimeria.

The compositions and methods of the invention may be useful for the treatment of an infection by a protozoan in a subject comprising administering to the subject an effective amount of a pharmaceutical composition comprising a first lipid synthesis inhibitor and a second lipid synthesis inhibitor. Alternatively, the compositions and methods of the invention may be useful for the study of the mechanism of action of lipid synthesis inhibitors and the synergistic activity of certain combinations of lipid synthesis inhibitors. It is envisioned that methods of the invention will also be useful for the rapid screening of putative candidate drugs as exemplified in Example 9 herein.

Typically, the EC50 maybe determined in vitro by calculating the concentration of the agent needed to inhibit growth of the protozoan by 50% compared to a control with no agent. Herein, the term “agent” includes an inhibitor, especially a lipid synthesis inhibitor. Protozoans may be grown in vitro by methods commonly known in the art. Growth, and subsequent inhibition of growth, may be determined by counting cell number both in the presence and absence of an anti-protozoal agent. Methods of counting and determining cell number are well known in the art.

Alternatively, the EC50 may be determined in vivo. Growth, and subsequent inhibition of growth, may be measured in vivo by methods commonly known in the art. Typically, such a method would comprise determining the number of protozoans in an infected host that was administered an anti-protozoal agent or combination of agents in comparison to the number of protozoans in an infected host that did not receive an anti-protozoal agent or combination of agents (i.e., a control host).

The present invention encompasses compositions where the ratio of EC50₁ to EC50_{c} is different than the ratio of EC50₂ to EC50_{c}, as long as each ratio is ten or greater. In one embodiment, the ratio of EC50₁ to EC50_{c} or the ratio of EC50₂ to EC50_{c} is greater than 20, 30, or 40, more preferably greater than 50, 60, 70, 80, 90, 100, or even more preferably greater than 150, 200, 250, 300, 400, 500, 600, or 1000.

Generally speaking, the first agent and the second agent that comprise a combination composition of the invention may be a chemical, biomolecule, or analyte such that the ratio of EC50₁ to EC50_{c} is ten or greater, and the ratio of EC50₂ to EC50_{c} is ten or greater. In some embodiments, the first agent and/or the second agent may be a biomolecule. Non-limiting examples of biomolecules include proteins, protein fragments, including individual amino acids, nucleic acids, including DNA and RNA, nucleic acid fragments, lipids, hormones, carbohydrates, or any combination of the above, i.e. a glycoprotein.

In other embodiments, the first agent and/or the second agent of a composition of the invention may be a chemical. Non-limiting examples of suitable chemicals are pharmaceutical compounds, enzyme inhibitors, or biomolecule mimics. Pharmaceutical compounds may include both FDA approved and non-approved drugs. Enzyme inhibitors may include both reversible and irreversible inhibitors. Non-limiting examples of reversible inhibitors may include competitive inhibitors, non-competitive inhibitors, uncompetitive inhibitors, and mixed inhibitors. Enzyme inhibitors may also refer to a biomolecule or chemical that decreases transcription or translation of the enzyme. Biomolecule mimics include peptide DNA, locked DNA, or other variants of polypeptides or nucleic acids.

In certain embodiments, the first or second agent may be an analyte. Non-limiting examples of analytes include a ligand, a chemical moiety, a compound, an ion, a salt, a metal, a secondary messenger of a cellular signal transduction pathway, a nanoparticle, an environmental contaminant, or a toxin.

The first agent may be a biomolecule, while the second agent is selected from the group comprising a biomolecule, a chemical, or an analyte. Alternatively, the first agent may be a chemical, while the second agent is selected from the group comprising a biomolecule, a chemical, or an analyte. In another alternative, the first agent may be an analyte, while the second agent is selected from the group comprising a biomolecule, a chemical, or an analyte. In each of the above examples, the first and second agent inhibit protozoan growth in an additive fashion, such that the ratio of EC50₁ to EC50_{c} is ten or greater, and the ratio of EC50₂ to EC50_{c} is ten or greater.

In one embodiment of the invention, the first or second agent may be a chemical or biomolecule that is a lipid synthesis inhibitor. Generally speaking, preferred compositions of the invention inhibit protozoan growth with minimal impact on the host. Therefore, the preferred lipid synthesis inhibitors of the invention are specific for protozoan lipid synthesis, as opposed to host synthesis. Non-limiting examples of suitable lipid synthesis inhibitors include the following classes: ergosterol synthesis inhibitors, sphingolipid synthesis inhibitors, and ether phospholipid inhibitors.

For example, the first or second agent may be an ergosterol synthesis inhibitor. Ergosterol is not produced by mammals. Therefore, ergosterol synthesis inhibitors typically will not interfere with host lipid synthesis. The biosynthesis of ergosterol from squalene is a multistep pathway comprising at least thirteen distinct enzymes. An ergosterol synthesis inhibitor may block the first enzyme in the pathway, squalene epoxidase. Examples of squalene epoxidase inhibitors include terbinafine, butenafine, and naftifine. The ergosterol synthesis inhibitor may block 14-delta-reductase. An example of a 14-delta-reductase inhibitor is amorolfine. The ergosterol synthesis inhibitor may be an azole compound that blocks lanosterol 14-alpha-demethylase. Suitable lanosterol 14-alpha-demethylase inhibitors include itraconazole, ketoconazole, clotrimazole, fluconazole, voriconazole, econazole, miconazole, oxiconazole, sulconazole, terconazole, tioconazole, posaconazole, and ravuconazole. Alternatively, the ergosterol synthesis inhibitor may block any other enzyme in the pathway, such as lanosterol synthase, C₄-methyloxidase, C₄-decarboxylase, 3-ketoreductase, C₂₄-methyltransferase, C₈-isomerase, C₅-desaturase, d22-desaturase, and d24-reductase. In one embodiment, the ergosterol synthesis inhibitor may be itraconazole. In another embodiment, the ergosterol synthesis inhibitor may be ketoconazole. In all embodiments, however, the first and second agent inhibits protozoan growth as determined by a ratio of EC50₁ to EC50_{c} greater than ten, and a ratio of EC50₂ to EC50_{c} greater than ten.

Certain protozoa, such as Trypanosomes, do not synthesize sterols de novo, but instead, use a salvage pathway that produces ergosterols from available host lipids. Therefore, in some embodiments, the ergosterol synthesis inhibitor is an ergosterol salvage inhibitor.

In another embodiment, the first or second agent may be a sphingolipid synthesis inhibitor. Sphingolipds are classified into three groups: ceramides, sphingomyelins, and glycosphingolipids. As with the ergosterol synthesis inhibitors, the preferred lipid synthesis inhibitor is specific or selective for protozoan lipid synthesis, as opposed to host lipid synthesis. Herein, an inhibitor that is “specific” or “selective” is an inhibitor that has a measurably greater effect on the cells or metabolism of a protozoan than on the cells or metabolism of a subject. Such “specific” or “selective” inhibitors include agents that may adversely effect the cells or metabolism of a subject. But, such adverse effects are neither life-threatening or permanently deleterious to the subject within the range of doses envisioned for compositions and methods of the invention. Suitable sphingolipid synthesis inhibitors may block any essential step in the synthetic pathway. One example is serine palmitoyltransferase, which is the first enzyme in the de novo synthesis of ceramide. An example of a serine palmitoyltransferase inhibitor is myriocin. A second example is ceramide synthase, which can be inhibited by fumonisin B., Alternatively, a sphingolipid synthesis inhibitor may block the last step in the synthesis of complex sphingolipids such as inositol phosphorylceramide (IPC) or glycosyl-ceramides. IPC is a predominate sphingolipid in protozoa. Thus, a sphingolipid synthesis inhibitor may inhibit IPC synthase, which catalyzes the transfer of phosphoinositol from phosphatidylinositol to ceramide to form IPC. Inhibitors of fungal IPC synthase include aureobasisin A, galbonolide A, khafrefungin, and rustmicin. Suitable IPC synthase inhibitors may include compounds that are specific for protozoan IPC synthase, for instance, a Leishmania IPC synthase inhibitor, a Plasmodium falciparum IPC synthase inhibitor, or a Toxoplasma IPC synthase inhibitor.

Certain protozoa, such as Leishmania, only produce IPC de novo during specific stages of their lives. In other protozoa and/or life stages, a salvage pathway is used to acquire precursors required for sphingolipid synthesis from available host lipids. Therefore, in some embodiments, the sphingolipid synthesis inihibitor may block the salvage pathway of synthesis. Inhibition may be mediated by blocking the salvage of sphingoid bases such as sphinganine, sphingosine or 3-keto-dihydrosphingosine, ceramides, or complex sphingolipids such as glycosyl-ceramides or sphingomyelin, singly or in combination. In each of the above embodiments, however, the combination of the first and second agent inhibits protozoan growth as determined by a ratio of EC50₁ to EC50_{c} that is ten or greater, and a ratio of EC50₂ to EC50_{c} that is ten or greater.

In an alternative embodiment, a lipid synthesis inhibitor may inhibit ether phospholipid synthesis. For instance, an inhibitor of ether phospholipid synthesis may block alklyl-dihydroxyacetone phosphate transferase, the first enzyme in the biosynthetic pathway. An inhibitor of ether phospholipid synthesis may also block the next enzyme in the pathway, alklyl-dihydroxyacetone phosphate reductase. An exemplary ether phospholipid synthesis inhibitor is specific for protozoan ether phospholipid synthesis, as opposed to host ether phospholipid synthesis.

Typically, both the first and the second lipid synthesis inhibitors are selected from different classes of lipid synthesis inhibitors. For instance, if the first agent is an ergosterol synthesis inhibitor, than the second agent may be a sphingolipid synthesis inhibitor or an ether phospholipid synthesis inhibitor. Alternatively, if the first agent is a sphingolipid synthesis inhibitor, than the second agent may be an ergosterol synthesis inhibitor or an ether phospholipid synthesis inhibitor. In another alternative, if the first agent is an ether phospholipid synthesis inhibitor, than the second agent may be an ergosterol inhibitor or a sphingolipid synthesis inhibitor. Preferred ergosterol synthesis inhibitors include itraconazole and ketoconazole, especially in combination compositions where the second agent may be a sphingolipid synthesis inhibitor or an ether phospholipid synthesis inhibitor.

In some embodiments, the first agent and the second agent may affect the integrity or stability of organized membrane domains. For instance, the first and the second agent may work together to disrupt the detergent resistant membrane fractions. Assays for detecting the disruption of detergent resistant membrane fractions are well known in the art, and a detailed protocol of such an assay is presented in the Examples below.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The application contains at least one drawing executed in color. Copies of this patent application publication with color drawing(s) will be provided by the Office upon request and payment of the necessary fee.

FIG. 1 shows that SL-free (spt2⁻) and ether phospholipid-free (EPL-free) (ads1⁻) promastigotes are hypersensitive to ITZ. FIG. 1A: Log phase L. major LV39 WT (in the absence and presence of 10 μM MYR), spt2⁻, and spt2⁻/+SPT2 promastigotes were grown in various concentrations of ITZ (0.01 nM-60 μM) and the effect of ITZ on growth was assessed relative to control cells grown in the absence of ITZ. FIG. 1B: Effect of myriocin (MYR) on the growth of LV39 WT promastigotes in the absence and presence of 0.5 μM ITZ. FIG. 1C: Using combinations of ITZ (0-1.05 μM) and MYR (0-55 μM), EC50s in LV39 WT promastigotes were determined and plotted in a classical isobologram. The fractional inhibitory concentration (FIC) was calculated as described in the Examples and plotted. FIGS. 1D-1E: Effects of ketoconazole (FIG. 1D) and clotrimazole (FIG. 1E) on the growth of LV39 WT and spt2⁻ promastigotes are shown. FIG. 1F: Effect of ITZ on the growth of LV39 WT and spt2⁻ promastigotes in the absence and presence of 200 mM EtN or choline. FIG. 1G: Effect of ITZ on the growth of LV39 WT and spt2⁻ promastigotes overexpressing putative L. major C14 demethylase (WT SSU::C14DM and spt2⁻ SSU::C14DM). FIGS. 1H and 1I: Effects of ITZ (FIG. 1H) and MYR (FIG. 1I) on the growth of FV1 WT and ads1⁻ promastigotes. Each experiment was repeated at least 3 times and results from one representative set are shown.

FIG. 2 shows that ITZ disrupts DRM rafts in LV39 WT and spt2⁻ promastigotes. Log phase L. major LV39 WT (FIG. 2A) and spt2⁻ (FIG. 2B) promastigotes were grown in various concentrations of ITZ (0-1.0 μM). After 48 hours, detergent resistance membrane (DRM) fractions were isolated at 4° C. and 37° C., as described in the Examples, followed by SDS-PAGE/western blot with α-LmGP63 mAb #235. Results are depicted in FIGS. 2A and 2B. Percentages of GP63 from insoluble (I) and soluble (S) fractions were determined using a Fuji Phosphoimager and are indicated below the images.

FIG. 3 shows the effect of ITZ and CLT on growth, sterol composition and DRM in LV39 WT and spt2⁻ promastigotes. Log phase L. major LV39 WT and spt2⁻ promastigotes were grown in various concentrations of ITZ (FIGS. 3A-3C) or CLT (FIGS. 3D-3F). After 48 hours, effects of ITZ and CLT on growth were assessed relative to control cells grown in the absence of any drugs (FIGS. 3A and 3D). Total lipids were isolated and analyzed by GC/MS and percentages of 14-methylfecosterol among all sterols are indicated in FIGS. 3B and 3E. Effects of ITZ and CLT on DRM were determined by analyzing the percentage of insoluble GP63 after detergent extraction at 4° C. and indicated in FIGS. 3C and 3F. Each experiment was repeated 2-3 times and error bars represent standard deviations.

FIG. 4 illustrates that ITZ disrupts the flagellar localization of FCaBP in spt2− promastigotes (FIG. 4A-4D). Log phase L. major LV39 WT and spt2-promastigotes transfected with pXGPhleo-FCaBP-HA were grown in the absence (FIGS. 4A and 4C) or presence (FIGS. 4B and 4D) of 0.2 mM ITZ. After 20 hours, cells were fixed and co-stained with α-HA mAb and rabbit α-PFR polyclonal antiserum as described in the Materials and Methods for the Examples. (FIG. 4E) Log phase promastigotes grown in 0.2 μM ITZ, 0.2 μM ITZ+200 mM EtN, or 3.5 mM ITZ were subjected to immunofluorescence assay as described. In each experiment, 150-200 cells were randomly selected and the percentage of cells showing the correct flagellar localization of FCaBP-HA was recorded. Note that many cells (˜50%) did not have enough signals from FCaBP-HA to be recorded; therefore, the percentage of cells showing flagellar localization was lower than 50%. Averages of 2-3 experiments are shown and error bars represent standard deviations.

FIG. 5 (FIG. 5) depicts an alignment of the P450-dependent C14DM genes from Leishmania major (systemic ID: LmjF11.1100 and accession number CAJ02958) (SEQ ID NO: 1), Homo sapiens (accession number Q16850) (SEQ ID NO: 2), Asperigillus fumigatus (accession number XP_{—}752137) (SEQ ID NO: 3), Candida albicans (accession number XP_{—}716822 XP_{—}437553) (SEQ ID NO: 4) and Mycobacterium tuberculosis (accession number NP_{—}215278) (SEQ ID NO: 5). Alignment was performed using the Clustal IW algorithm included in the Meglign program (Laser Gene) with conserved regions highlighted. Asterisks represent amino acids that are replaced (G49R, Y115H, and S382F) in LmC14DM.

FIG. 6 (FIG. 6) illustrates that overexpression of LmC14DM confers moderate resistance to ITZ in spt2− (FIG. 6B), but not in WT (FIG. 6A) parasites. Unmodified or modified forms of LmC14DM genes were integrated into the small ribosomal subunit site of WT or spt2⁻ promastigotes to achieve overexpression, and the susceptibility of transfectants was determined as described. C14DM** contains mutations G49R and Y115H, and C14DM*** contains mutations G49R, Y115H, and S382F (see FIG. 5).

### DETAILED DESCRIPTION

The present invention provides methods and compositions useful for inhibiting the growth of protozoans. Additionally, compositions of the present invention may be used to treat protozoan infections and/or disorders relating to a protozoan infection; or alternatively, the compositions may be useful for the study of lipid synthesis inhibitors and the methods used for the rapid screening of putative drugs or target sites.

### I. Compositions for Inhibiting Protozoan Growth

A. Combinations of Lipid Synthesis Inhibitors

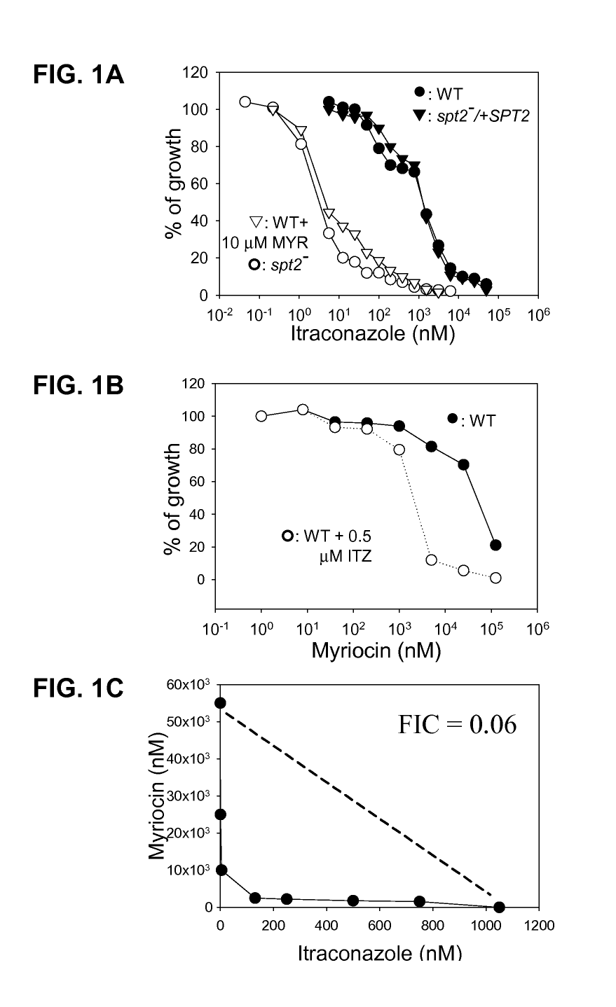

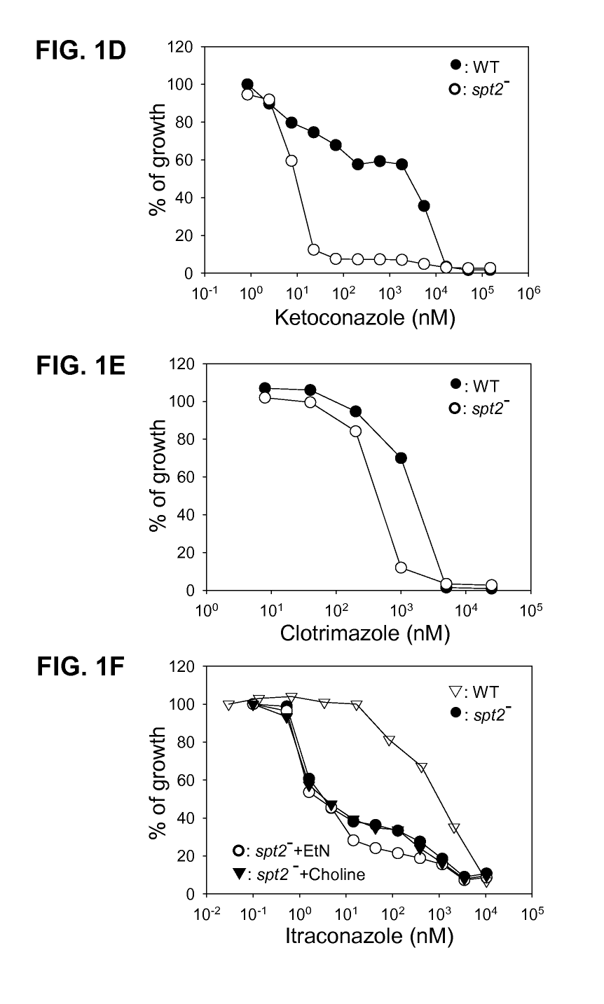

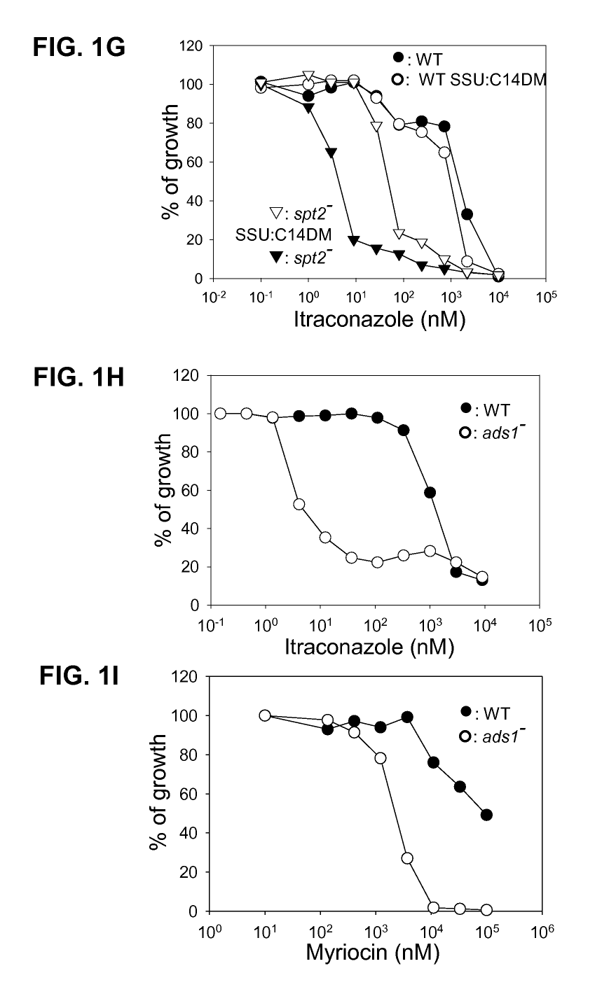

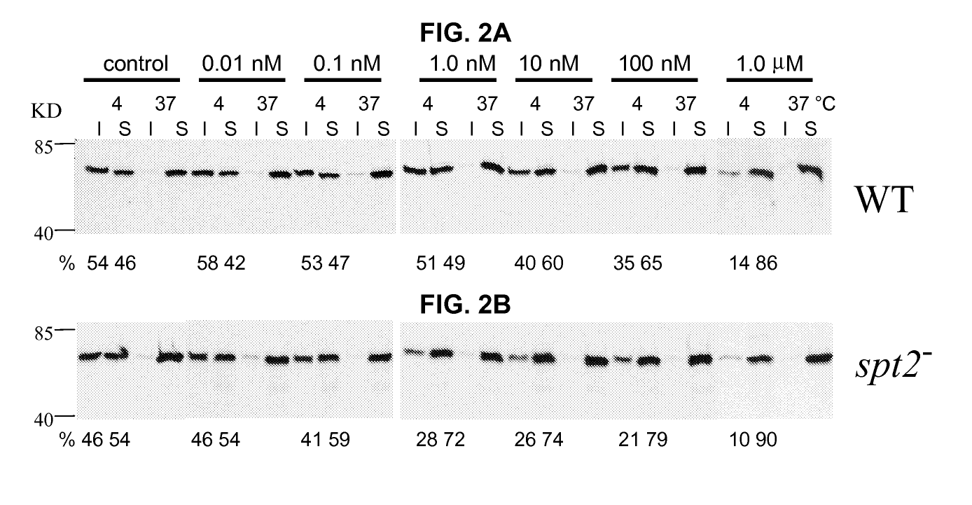

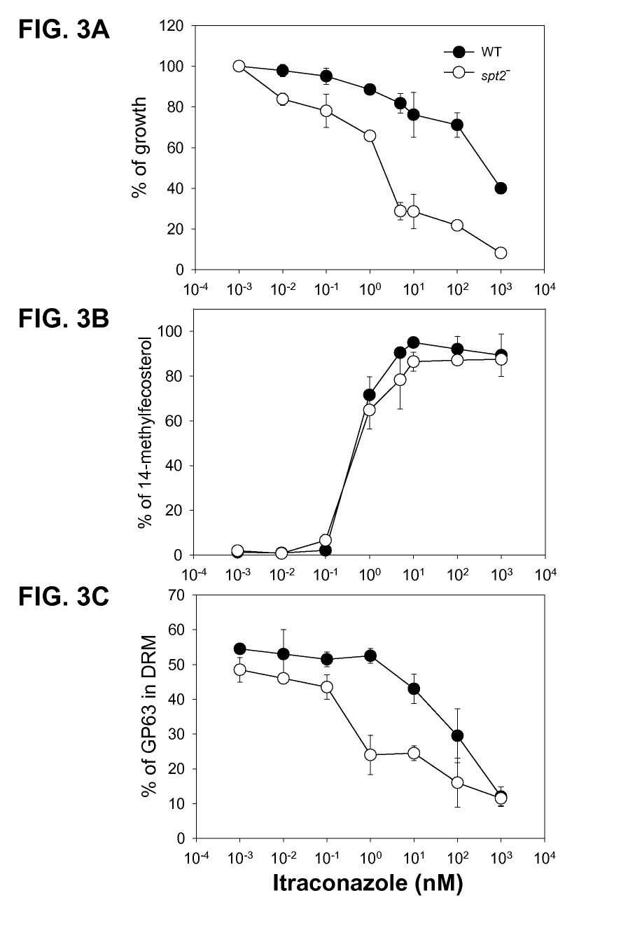

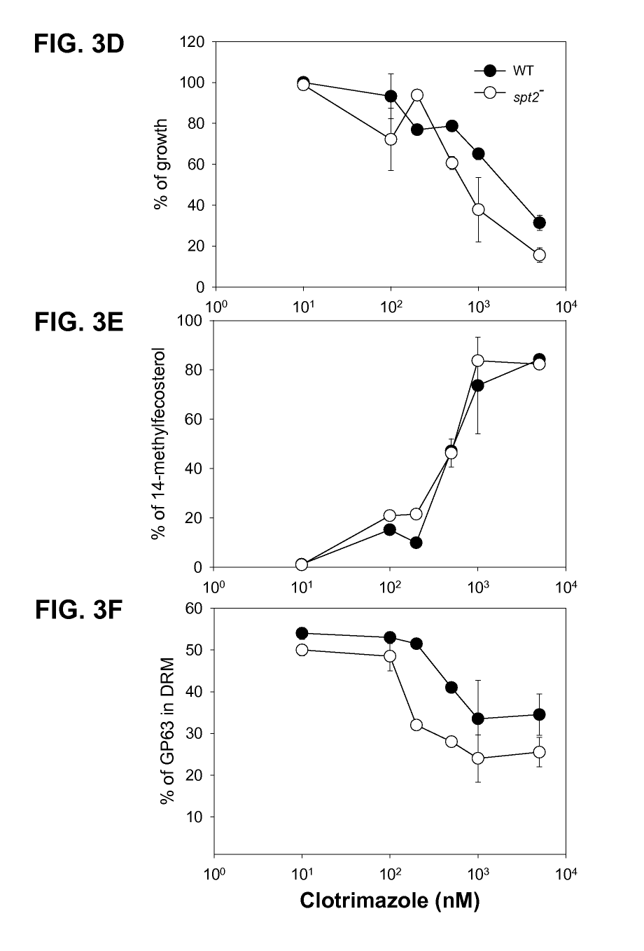

The combination of MYR and ITZ synergistically inhibits the growth of L. major promastigotes (FIG. 1C). The effect from MYR is clearly due to inhibition of SL (by shutting down serine palmitoyltransferase, [Zhang, et al., 2003]), suggesting SLs serve pivotal functions besides ethanolamine biosynthesis (FIG. 1F) [Zhang, 2007]. Targets of ITZ, however, are not as clear. In fungi, the primary target of imidazole and triazole derivatives such as fluconazole, ITZ and KEZ is the cytochrome P450-dependent lanosterol 14alpha-demethylase (C14DM). Treatments with these azoles lead to depletion of ergosterol and accumulation of 14-methylated sterols [Yoshida, 1988; Vanden Bossche, et al., 1987]. A similar mechanism of action has been proposed for the anti-leishmanial effect of these compounds [Hart, et al., 1989; Berman, et al., 1984], although alternative modes of action were also suggested [Lira, et al., 2001]. Here it is shown that 5-10 nM of ITZ or KEZ completely altered the sterol composition, yet had only minor effect on growth in LV39 WT promastigotes (FIG. 3; Table 1). Therefore, inhibition of LmC14DM by itself is not sufficient to cause severe growth retardation in L. major promastigotes. At concentrations higher than 1 mM, ITZ disrupted DRMs (FIGS. 2, 3) and caused substantial inhibition of growth, suggesting that: 1) proper maintenance of membrane microdomains (which was reflected by DRMs) is essential for Leishmania growth; and 2) at higher concentrations (>1 mM), ITZ affects additional targets (e.g. other membrane lipids) that are important for the maintenance of membrane microdomains. Furthermore, the fact that ITZ did not drastically reduce the abundance of total sterol species in Leishmania parasites, rather than replaced the endogenous ergosterols with 14-methylated sterols, suggests the extra 14-methyl group may hinder the ability to stabilize DRMs.

TABLE 1
Effects of lipid metabolism inhibitors on the growth of WT and spt2⁻ promastigotes.

| | | EC50 in | EC50 in | EC50 (WT)/ |
|---|---|---|---|---|
| Inhibitor | Putative target/mechanism | WT | spt2⁻ | EC50 (spt2⁻) |
| Itraconazole | Lanosterol 14α-demethylase | 1.1 μM | 3.9 nM | 282 |
| Ketoconazole | Lanosterol 14α-demethylase | 3.1 μM | 10 nM | 310 |
| Miconazole | Lanosterol 14α-demethylase | 4.3 μM | 0.70 μM | 6.1 |
| Fluconazole | Lanosterol 14α-demethylase | 42 μM | 17 μM | 2.5 |
| Clotrimazole | Lanosterol 14α-demethylase | 1.5 μM | 0.65 μM | 2.3 |
| 3-(Biphenyl-4-yl)-3- | Squalene synthase | 1.58 μM | 1.12 μM | 1.4 |
| hydroxyquinuclidine | | | | |
| (BPQ-OH) | | | | |
| 22,26-azalsterol | Δ²⁴⁽²⁵⁾-sterol methyltransferase | 0.63 μM | 63 nM | 10 |
| Mevinolin | HMG-CoA reductase | 15 μM | 15 μM | 1.0 |
| Terbinafine | Squalene epoxidase | 4.3 μM | 3.3 μM | 1.3 |
| Aureobasidin A | IPC synthase | 0.56 μM | 0.62 μM | 0.90 |
| Miltefosine | CTP: phosphocholine cytidyl | 21 μM | 22 μM | 0.95 |
| | transferase, anti-proliferative | | | |
| | lysophospholipid analogs | | | |
| Edelfosine | CTP: phosphocholine cytidyl | 1.5 μM | 5.4 μM | 0.28 |
| | transferase, anti-proliferative | | | |
| | lysophospholipid analogs | | | |
| Amphotericin B | Binds to ergosterol/sterol; | 41 nM | 22 nM | 1.9 |
| | interferes with membrane | | | |
| | permeability | | | |
| Cinnamycin | Binds to phosphatidyl- | 3.1 μM | 3.1 μM | 1.0 |
| | ethanolamine and induces | | | |
| | cytolysis | | | |

Log phase L. major LV39 WT and spt2 promastigotes were inoculated at 1.0×10⁵ cells/ml and culture densities were determined 48 hours later. For each compound, the concentration required to inhibit 50% of growth (EC50) was determined as described in Materials and Methods. Each experiment was repeated at least 3 times and results from one representative set were shown.

In contrast to WT parasites, nanomolar concentration of ITZ was sufficient to cause disruption of DRM and inhibit growth in the SL-free spt2⁻ mutants (FIGS. 2, 3), suggesting that in the absence of SLs, parasites are much more dependent upon ergosterol synthesis to maintain essential membrane microdomains. Interestingly, other azole drugs that were tested including CLT, miconazole, and fluconazole did not show such selective activity against spt2⁻ mutants (Table 1). One possibility is that these azoles need micromolar concentrations (instead of nanomolar concentrations for ITZ/KEZ) to inhibit LmC14DM in Leishmania and at such conditions they have additional targets besides LmC14DM therefore affect WT and spt2− parasites nearly equally well. Similarly, inhibitors of other enzymes in the sterol synthesis pathway did not show strong selectivity against spt2− parasites, which indicates targets beyond sterol synthesis are involved (Table 1). In addition, the extreme hypersensitivity of spt2⁻ mutants was only partially reversed through the overexpression of LmC14DM, the bona fide target of ITZ in fungi (FIG. 1G), suggesting other target(s) or mechanisms are involved.

Although SL and sterol molecules are known to be enriched in DRMs and promote the formation of rafts, it is quite remarkable that genetic (sp2−) or chemical (10 μM MYR treatment) disruption of SL biosynthesis results in almost 300-fold reduction in EC50 to ITZ and KEZ (Table 1). The ether phospholipid-null mutant ads1⁻ was hypersensitive to both SL inhibitor (MYR) and ergosterol synthesis inhibitors (ITZ/KEZ). These results lead to the proposal that ergosterols, IPC (SLs), and ether phospholipids form extensive interactions among each other to stabilize membrane microdomains. Removal of one class of molecules (SLs or ergosterols) is not sufficient to disrupt these microdomains probably due to the stabilizing power from the other components and any compensatory effects. Yet it would leave cells extremely vulnerable to perturbations of the other two components. Although the exact mechanism of such synergistic effect is not clear, current data indicate it correlates with compromised trafficking of a subset of raft-associated proteins.

Regardless of the mechanism, the synergistic effect of this strategy (with a FIC of 0.06, FIG. 1C) could have important therapeutic implications. One thing to keep in mind is that MYR, a potent inhibitor of serine palmitoyltransferase, is unlikely to shut down the SL biosynthesis in amastigotes, the intracellular form of Leishmania parasites. This is because unlike promastigotes, which synthesize IPC de novo via serine palmitoyltransferase, the majority of IPC in intracellular amastigotes is made through salvage of sphingoid bases and/or ceramides from the host [Zhang, et al., 2005]. Indeed, preliminary results indicate MYR does not improve the efficacy of ITZ on amastigotes growing in murine macrophages (data not shown). To circumvent this problem, one solution is to use an inhibitor of IPC synthase instead of MYR since it should affect both promastigotes and amastigotes yet be safe to hosts (since mammals do not make IPC). However, the fungal IPC synthase inhibitor aureobasidin A did not show any selectivity against spt2⁻ parasites, and preliminary results suggested that growth inhibition by this compound was not simply related to inhibition of IPC synthesis in promastigotes (data not shown). Therefore, more potent and specific inhibitors of Leishmania IPC synthase are needed. Alternatively, agents that cause blockage of SL salvage from host should also hold promise to enhance the efficacy of ITZ in amastigotes.

B. Selecting Lipid Synthesis Inhibitors

Generally speaking, an agent of the invention may be selected by exposing a protozoan to a first agent, and then screening potential agents to find a second agent that inhibits protozoan growth such that the ratio of EC50₁ to EC50_{c} is ten or greater, and the ratio of EC50₂ to EC50_{c} is ten or greater. The assay is described in Mackey et al., “Discovery of Trypanocidal Compounds by Whole Cell HTS of Trypanosoma brucei,” Chem Biol Drug Des (2006) 67(5):355-63, hereby incorporated by reference, describes an assay that may be used to select a first and second agent for inhibiting Trypanosoma growth. The assay can readily be modified by those skilled in the art to find inhibitors of other protozoans. Potential agents that may be screened include biomolecules, chemicals, and analytes. An exemplary screening library would include FDA approved drugs.

One of ordinary skill in the art, after examining the lipid composition of the target protozoan's cell membrane, would know what class or classes of lipid synthesis inhibitors to screen for potential agents. For instance, Leishmania does not produce sphingolipids de novo during their amastigote life stage. Instead, they utilize a salvage pathway. Therefore, one of ordinary skill in the art would know that sphingolipid salvage inhibitors could be screened for potential anti-Leishmania agents. Similarly, one of ordinary skill would recognize that trypanosomes salvage sterols from their host. Therefore, potential anti-Trypanosome agents include sterol salvage inhibitors. In particular, one of ordinary skill in the art would appreciate that exemplary potential agents would target protozoan lipid synthesis pathways while not disrupting host lipid synthesis pathways. If the lipid composition of a particular protozoan is unknown, then the genome of the protozoan may be examined to help determine which lipid synthesis pathways are present in a particular protozoan.

Alternatively, first and second agents may be selected that disrupt detergent resistant membrane domains, i.e. lipid rafts. Typically, a first agent known to disrupt detergent resistant membrane domains may be selected while potential agents are screened for a second agent, such that the ratio of EC50₁ to EC50_{c} is ten or greater, and the ratio of EC50₂ to EC50_{c} is ten or greater. Assays for determining the disruption of detergent resistant membrane rafts are well known in the art. One such assay is detailed in the Examples below.

C. Protozoan Growth Inhibited

A composition of the present invention may inhibit growth of a wide range of protozoans. In one embodiment, a composition inhibits the growth of Leishmania spp. Examples of Leishmania species include Leishmania aethiopica, Leishmania amazonensis, Leishmania arabica, Leishmania archibaldi, Leishmania aristedesi, Leishmania (Viannia) braziliensis, Leishmania chagasi (syn. Leishmania infantum), Leishmania (Viannia) colombiensis, Leishmania deanei, Leishmania donovani, Leishmania enriettii, Leishmania equatorensis, Leishmania forattinil, Leishmania garnhami, Leishmania gerbili, Leishmania (Viannia) guyanensis, Leishmania herreri, Leishmania hertigi, Leishmania infantum, Leishmania killicki, Leishmania (Viannia) lainsoni, Leishmania major, Leishmania mexicana, Leishmania (Viannia) naiffi, Leishmania (Viannia) panamensis, Leishmania (Viannia) peruviana, Leishmania (Viannia) pifanoi, Leishmania (Viannia) shawi, Leishmania tarentolae, Leishmania tropica, Leishmania turanica, and Leishmania venezuelensis.

In another embodiment, a composition of the invention may inhibit the growth of Plasmodium spp. Examples of Plasmodium species include Plasmodium berghei, Plasmodium brasilianum, Plasmodium chabaudi, Plasmodium cynomolgi, Plasmodium falciparum spp., Plasmodium gallinaceum, Plasmodium knowlesi, Plasmodium lophurae, Plasmodium malariae, Plasmodium ovale, Plasmodium relictum, Plasmodium vivax, and Plasmodium yoelii.

In yet another embodiment, a composition of the invention may inhibit the growth of a Trypanosoma. Examples of Trypanosoma species include T. avium, which causes trypanosomiasis in birds, T. boissoni, T. brucei, which causes sleeping sickness in humans and nagana in cattle, T. carassii, T. cruzi, which causes Chagas disease in humans, T. congolense, which causes nagana in cattle, horses, and camels, Trypanosoma equinum Voges 1901, T. equiperdum, which causes dourine or Covering sickness in horses and other Equidae, T. evansi, which causes one form of the disease surra in certain animals (human infection reported in 2005 in India), Trypanosoma lewisi, Trypanosoma melophagium, Trypanosoma percae, Trypanosoma rangeli, T. rotatorium in amphibian, T. simiae, which causes nagana in animals, T. suis, T. theileri, T. triglae in marine teleosts, and T. vivax, which causes the disease nagana.

In still yet another embodiment, a composition of the invention may inhibit the growth of Trichomonas, in particular, Trichomonas vaginalis. In an alternative embodiment, a composition of the invention may inhibit the growth of Entamoeba histolytica or Giardia lamblia. In another alternative, a composition of the invention may inhibit the growth of Eimeria, including, in particular, the species of Eimeria that infect chickens, for instance, Eimeria acervulina, Eimeria tenella, and Eimeria maxima. In still yet another alternative, a composition of the invention may inhibit the growth of a Sarcocystis species, particularly the species that infect humans, opposums, and horses.

In a further embodiment, a composition of the invention may inhibit the growth of Cryptosporidium species. Examples of Cryptosporidum species include C. parvum, C. hominis (previously C. parvum genotype 1), C. canis, C. felis, C. meleagridis, and C. muris.

In yet a further embodiment, a composition of the invention may inhibit the growth of a Thelileria species, including Theileria parva, T. annulata, T. taurotragi, T. mutans, T. velifera and T. orientalis. In certain embodiments, a composition of the invention may inhibit the growth of a Babesia species, including Babesia bovis, Babesia divergens, and Babesia microti.

### II. Method of Inhibiting Protozoan Growth

The present invention encompasses methods of inhibiting protozoan growth. The methods comprise contacting a protozoan with an effective amount of a composition comprising a first agent and a second agent, such that the ratio of the EC50 of the first agent alone to the EC50 of the composition is ten or greater, and the ratio of the EC50 of the second agent alone to the EC50 of the composition, is ten or greater. Stated in an alternative manner, methods of the invention comprise contacting a protozoan with an effective amount of a combination composition comprising two lipid synthesis inhibitors such that the EC50 of composition is at least 10-fold lower than the individual EC50s for either of the lipid synthesis inhibitors alone. More preferably, the method uses a combination composition having an EC50 of 20-fold, 50-fold, 100-fold, or even greater than a 1000-fold lower than the individual EC50s for either of the lipid synthesis inhibitors that comprise the combination.

Methods of contacting the protozoan with an effective amount of a composition are described in more detail below and in the examples. An effective amount of a composition, as used herein, means an amount necessary to produce the desired inhibition of growth.

Protozoan growth may be inhibited in vitro, as detailed in the examples, or in vivo, as detailed below.

### III. Method of Treating a Protozoan Infection

One aspect of the present invention encompasses a method of treating a protozoan infection in a subject. The method comprises administering to the subject an effective amount of a pharmaceutical composition comprising a first agent and a second agent, such that the ratio of the EC50 of the first agent alone to the EC50 of the composition is ten or greater, and the ratio of the EC50 of the second agent alone to the EC50 of the composition, is ten or greater. Stated alternatively, the EC50 of the combination composition of the first and second lipid synthesis inhibitors is at least 10-fold less than either of the individual EC50s for the first or second lipid synthesis inhibitor alone.

The invention permits the artisan to treat a subject having a protozoan infection or to provide treatment to inhibit a protozoan infection in a subject. Treatments include administering a therapeutically effective amount of a pharmaceutical composition of the invention. Thus, in some embodiments, a composition of the invention is administered to treat or inhibit infection in a subject. As used herein, the term “inhibit infection” refers to a prophylactic treatment that increases the resistance of a subject to infection with a protozoan or, in other words, decreases the likelihood that the subject will become infected with the protozoan.

By a “therapeutically effective amount” of a composition of the invention is meant a sufficient amount of the composition to treat protozoan infections, at a reasonable benefit/risk ratio applicable to any medical treatment. It will be understood, however, that the total daily usage of the compositions of the present invention will be decided by the attending physician within the scope of sound medical judgment.

The terms “treat,” “treated,” or “treating,” when used with respect to administration to a subject refers to a therapeutic regimen that decreases the amount or effect of an infectious agent in a subject who has become infected in order to fight the infection, e.g., reduce or eliminate the infection or prevent it from becoming worse, or which prevents a further increase in amount or activity of an infectious agent. The term “treating”, as used herein, unless otherwise indicated, means reversing, alleviating, inhibiting the progress of, or inhibiting the development of a disorder or condition or one or more symptoms of such disorder or condition caused by protozoan infection. The term “treatment”, as used herein, refers to the act of treating, as “treating” is defined immediately above.

The present method of treating a protozoan infection has broad application against many different protozoans, as described in section I above.

A. Subjects

As used herein, a “subject” shall mean a human, vertebrate, or invertebrate animal including but not limited to a dog, cat, ungulate, horse, cow, pig, sheep, opossum, goat, non-human primate (e.g. monkey), lagomorph, rabbit, rodent, rat, mouse, bird, arthropod (e.g. a tick) or insect (e.g. a mosquito, fly, or sand fly).

One class of subjects according to the present invention is subjects having a protozoan infection. Such subjects are subjects in need of treatment with a protozoan inhibitor. This class of subjects includes subjects diagnosed with infection, exhibiting symptoms of infection, or having been exposed to a protozoan. A subject at risk of developing a protozoan infection is a subject in need of prevention of infection. Such subjects include those at risk of exposure to an infection-causing protozoan. For instance, a subject at risk may be a subject who is planning to travel to an area where a particular type of infectious protozoan is found or it may be a subject who through lifestyle or medical procedures is exposed to bodily fluids which may contain a protozoan or even any subject living in an area that a protozoan has been identified. Subjects at risk of developing infection also include general populations to which a medical agency recommends pre-emptive infectious measures for a particular infectious organism. In addition, immunocompromised subjects (such as subjects with AIDS or undergoing cancer treatment) are at a disproportional high risk for infections by opportunistic pathogens such as Toxoplasma and Cryptosporidium.

Alternatively, a subject might be a carrier of a protozoan infection or a reservoir of infection. For instance, dogs are a known reservoir of certain Leishmania species, and opossums are a known reservoir for certain Sarcosystis species.

A subject may or may not exhibit symptoms of infection such as fever, swollen lymph glands, muscle aches, and pains. Methods to diagnose symptomatic and asymptomatic protozoan infection are known to those of ordinary skill in the medical arts and are described below herein. Some methods of diagnosis include, but are not limited to, blood tests for antibodies to the protozoal parasite and other assays such as lymph assays for protozoal parasites. Scans by computerized tomography (CT scan) or magnetic resonance imaging (MRI scan) may also be used in the diagnosis of some types of protozoal infection, for example brain scans for Toxoplasma infection.

B. Diagnostic Tests to Determine Protozoan Infection Status of a Subject

Diagnostic tests known to those of ordinary skill in the art may be used to assess protozoan infection status of a subject and to evaluate a therapeutically effective amount of the administered composition. Examples of diagnostic tests are set forth below. A determination of protozoan infection may be obtained using one of the methods described below (or other methods known in the art). If necessary, a secondary determination of protozoan infection may also be made. A comparison of the infection levels may be used to assess the effectiveness of administration of a composition of the invention as a prophylactic or a treatment of the protozoan infection. Absence of a protozoan infection may be an indication for prophylactic intervention by administering a composition described herein to prevent a protozoan infection.

An example of a method of diagnosis of acute Toxoplasma infection involves assessing the levels of parasites remaining in the blood after exposure. This may be accomplished by isolation of the parasite from either blood or other body fluids after subinoculation of the body fluid into the peritoneal cavity of mice. (see Harrison's Principles of Internal Medicine, 14/e, McGraw Hill Companies, New York, 1998). If no parasites are found in the mouse's peritoneal fluid, its anti-Toxoplasma serum titer can be evaluated 4 to 6 weeks after inoculation. The presence of Toxoplasma gondii in a subject's body fluid represents an acute infection, and the presence of Toxoplasma gondii in tissue biopsies is an indication only of the presence of tissue cysts and not acute toxoplasmosis. (see Harrison's Principles of Internal Medicine, 14/e, McGraw Hill Companies, New York, 1998). Additional methods of diagnosis and assessment of chronic and acute toxoplasma infection are known to those of skill in the art.

Those of ordinary skill in the art know tests useful for diagnosis of other protozoan infections. For example, diagnosis of malaria can be done by microscopic identification of asexual forms of the parasite in peripheral blood smears stained with Romanovsky staining, or Giemsa at pH 7.2, Wright's, Field's, or Leishman's stain. Both thin and thick blood smears may be examined. In addition, a finger-prick blood test is also available, in which the presence of P. falciparum histidine-rich protein 2 is determined. Additional methods of diagnosis and assessment of Plasmodium infection are known to those of skill in the art. The level of parisitemia may be important in the prognosis and can be determined with the above-identified diagnostic tests and by other means known in the art.

In addition to the diagnostic tests described above, clinical features of Plasmodium infection can be monitored for assessment of infection. These features include, but are not limited to: normochromic, nomocytic anemia, erythrocyte sedimentation rate, plasma viscosity, and platelet count may be reduced. Subjects may also have metabolic acidosis, with low plasma concentrations of glucose, sodium, bicarbonate, calcium, phosphate, and albumin together with elevations in lactate, blood urea nitrogen, creatinine, urate, muscle and liver enzymes, and conjugated and unconjugated bilirubin. In adults and children with cerebral malaria, the mean opening pressure at lumbar puncture is about 160 mm cerebrospinal fluid; the cerebrospinal fluid usually is normal or has a slightly elevated total protein level [<1.0 g/L (100 mg/dL)] (see Harrison's Principles of Internal Medicine, 14/e, McGraw Hill Companies, New York, 1998).

For Eimeria diagnosis, a lymph node biopsy smear and thick and thin blood films, may be performed.

A diagnostic procedure for Babesia may include examination of Giemsa-stained thick and thin blood films for small intraerythrocytic parasites. Babesia does not cause the production of pigment in parasites, nor are schizonts or gametocytes formed. An indirect immunofluorescence antibody test is useful for the diagnosis of infection with B. microti with serum antibody titer rising 2 to 4 weeks after the onset of illness and declining over 6 to 12 months. Another diagnostic assay involves the transfer of a bodily sample from a patient suspected of infection into a test animal. For instance, intraperitoneal inoculation of blood from patients with babesiosis into hamsters or gerbils results in detectable parasitemia within 2 to 4 weeks. (see Harrison's Principles of Internal Medicine, 14/e, McGraw Hill Companies, New York, 1998).

Sarcosporidiosis diagnosis may be based on the identification of sporocysts in the subject's stool or the identification of cysts measuring about 100 to 325 m in striated or cardiac muscle. Clinical symptoms may include muscle pain and swelling in humans. (see Harrison's Principles of Internal Medicine, 14/e, McGraw Hill Companies, New York, 1998). For horses, diagnosis may be based on the presence of Sarcosystis in cerebral spinal fluid. Clinical symptoms in horses may include asymmetric incoordination (ataxia), weakness and spasticity, lameness, airway abnormalities, such as laryngeal hemiplegia (paralyzed flaps), dorsal displacement of the soft palate (snoring), or airway noise of undetermined origin, a slight gait asymmetry of the rear limbs, focal muscle atrophy, or even generalized muscle atrophy, loss of condition, upward fixation of the patella (locking up of the stifle), or back soreness, which can be severe.

Cryptosporidium diagnosis includes fecal examination for small oocysts, which are 4 to 5 m in diameter and are smaller than the fecal stages of most other parasites. Detection may be enhanced by techniques including modified acid-fast and direct immunofluorescent stains and enzyme immunoassays. If low numbers of oocysts are being excreted, Sheather's coverslip flotation method concentrates them for examination. Cryptosporidia also can be identified by light and electron microscopy at the apical surfaces of intestinal epithelium from biopsy specimens of the small bowel and, less frequently, the large bowel. (see Harrison's Principles of Internal Medicine, 14/e, McGraw Hill Companies, New York, 1998).

Diagnosis of Theileria may be done via identification of schizonts in superficial lymph nodes or spleen, using serodiagnosis, and/or the identification of piroplasms coincident with fever.

Methods of diagnosing leishmaniasis are well known in the art and include microscopically identifying amastigotes in tissue samples, performing a PCR reaction with species specific PCR primers, or performing cellulose acetate electrophoresis. There are several possible Leishmania infections, including localized cutaneous leishmaniasis, diffuse cutaneous leishmaniasis, recidivans cutaneous leishmaniasis, PKADL, MCL, and VL. Symptoms vary with the type of infection, but may include nonspecific ulcers, and in severe cases wasting, massive splenomegaly, pancytopenia, hypergammaglobulinemia, and intermittent fevers.

The identification of protozoa in or on an object, may be performed via standard diagnostic methods described above including microscopic examination, antibody labeling in a sample of the object, and by PCR analysis of a sample.

C. Pharmaceutical Compositions of the Invention

It will be appreciated that a composition of the present invention may exist, where appropriate, as a pharmaceutical composition suitable for administration to a subject. According to the present invention, a pharmaceutical composition includes, but is not limited to, pharmaceutically acceptable salts, esters, salts of such esters, or any other adduct or derivative which upon administration to a subject in need is capable of providing, directly or indirectly, a composition as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and other subjects without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 119 (1977), incorporated herein by reference. The salts can be prepared in situ during the final isolation and purification of the composition of the invention, or separately by reacting the free base function with a suitable organic acid. Non-limiting examples of pharmaceutically acceptable, nontoxic acid addition salts are salts of an amino group formed with inorganic acids such as hydrochloric acid, hydrobromic acid, hydroionic acid, nitric acid, carbonic acid, phosphoric acid, sulfuric acid and perchloric acid.

Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, oxalic, malonic, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Representative alkali or alkaline earth metal salts include sodium, lithium, potassium, calcium, magnesium, aluminum, zinc and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Additionally, organic salts made from N,N′-dibenzylethylenediamine, chloroprocaine, choline, diethanolamine, ethylenediamine, meglumine-(N-methylglucamine) and procaine may be appropriate. All of these salts may be prepared by conventional means from the corresponding compound by reacting, for example, the appropriate acid or base with the any of the agents of the invention.

Additionally, as used herein, the term “pharmaceutically acceptable ester” refers to esters, which hydrolyze in vivo and include those that break down readily in the subject's body to leave the parent agent or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butyrates, acrylates and ethylsuccinates.

Furthermore, the term “pharmaceutically acceptable prodrugs” as used herein refers to those prodrugs of the agents of the present invention which are, within the scope of sound medical judgment, suitable for use in contact with the tissues of humans and other subjects with undue toxicity, irritation, allergic response, and the like, commensurate with a reasonable benefit/risk ratio, and effective for their intended use, as well as the zwitterionic forms, where possible, of the agents of the invention. The term “prodrug” refers to agents that are rapidly transformed in vivo to yield the parent agent of the above compositions, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

The pharmaceutical compositions of the present invention may additionally comprise a pharmaceutically acceptable carrier, which, as used herein, means a non-toxic, inert solid, semi-solid or liquid filler, diluent, encapsulating material or formulation auxiliary of any type. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

D. Methods of Administering a Therapeutically Effective Dose

The agents of the present invention can be formulated into pharmaceutical compositions and administered by a number of different means that will deliver a therapeutically effective dose. Such compositions can be administered orally, parenterally, by inhalation spray, rectally, intradermally, intracisternally, intravaginally, intraperitoneally, transdermally, bucally, as an oral or nasal spray, or topically (i.e. powders, ointments or drops) in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Liquid dosage forms for oral administration include, but are not limited to, pharmaceutically acceptable emulsions, microemulsions, solutions, suspensions, syrups and elixirs. In addition to the active agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, dextrose, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. Dimethyl acetamide, surfactants including ionic and non-ionic detergents, and polyethylene glycols may also be used. Mixtures of solvents and wetting agents such as those discussed above are also useful.

The injectable formulations (aqueous or non-aqueous isotonic sterile injection solutions or suspensions) may be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissues.

Compositions for rectal or vaginal administration are preferably suppositories which can be prepared by mixing the agents of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the composition is ordinarily combined with one or more adjuvants appropriate to the indicated route of administration. If administered per os, the composition can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Additionally, the active agent may be mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active agents may also be in micro-encapsulated form with one or more excipients as noted above. The solid dosage forms of tablets, dragees, capsules, pills, and granules may be prepared with coatings and shells such as enteric coatings, release controlling coatings and other coatings well known in the pharmaceutical formulating art. In such solid dosage forms the active composition may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and may also be of a composition that they release the active ingredient(s) only, or preferentially, in a certain part of the intestinal tract, optionally, in a delayed manner. Examples of embedding compositions that may be used include polymeric substances and waxes. Dosage forms for topical or transdermal administration of an agent of this invention include ointments, pastes, creams, lotions, gels, powders, solutions, sprays, inhalants or patches. The active agent is admixed under sterile conditions with a pharmaceutically acceptable carrier and any needed preservatives (e.g., anti-microbials, anti-oxidants, chelating agents, and inert gases and the like) or buffers as may be required. Ophthalmic formulation, ear drops, and eye drops are also contemplated as being within the scope of this invention.

The ointments, pastes, creams and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to the agents of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a composition to the body. Such dosage forms can be made by dissolving or dispensing the composition in the proper medium. Absorption enhancers can also be used to increase the flux of the composition across the skin. The rate can be controlled by either providing a rate controlling membrane or by dispersing the composition in a polymer matrix or gel.

The composition may be prepared in various forms for administration, including tablets, caplets, pills or dragees, or can be filled in suitable containers, such as capsules, or, in the case of suspensions, filled into bottles. As used herein, “pharmaceutically acceptable carrier medium” means a non-toxic material that does not interfere with the effectiveness of the biological activity of the active ingredients. The characteristics of the carrier will depend on the route of administration. Pharmaceutically acceptable carrier mediums include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-protozoan compositions of the invention, such as by producing any undesirable biological effect or otherwise interacting in a deleterious manner with any other agent(s) of the pharmaceutical composition, its use is contemplated to be within the scope of this invention. Organic or inorganic solid or liquid carrier media suitable for enteral or parenteral administration can be used to make up the composition. Gelatine, lactose, starch, magnesium, stearate, talc, vegetable and animal fats and oils, gum, polyalkylene glycol, or other known carriers for medicaments may all be suitable as carrier media.

E. Effective Dose

The compositions of the invention may be administered using any amount and any route of administration effective for attenuating infectivity of the protozoan. Thus, the expression “amount effective to attenuate infectivity of a protozoan”, as used herein, refers to a nontoxic but sufficient amount of the anti-protozoal agent to provide the desired treatment of protozoan infection. The exact amount required will vary from subject to subject, depending on the species, age, and general condition of the subject, the severity of the infection, the particular anti-protozoan agent, its mode of administration, and the like. The anti-protozoal compositions of the invention are preferably formulated in dosage unit form for ease of administration and uniformity of dosage. The expression “dosage unit form” as used herein refers to a physically discrete unit of anti-protozoan agent appropriate for the patient to be treated. Each dosage should contain the quantity of active material calculated to produce the desired therapeutic effect either as such, or in association with the selected pharmaceutical carrier medium.

The amount of the composition of the invention that can be combined with the carrier materials to produce a single dosage of the composition will vary depending upon the patient and the particular mode of administration. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

F. Evaluating Activity of the Pharmaceutical Compositions

An in vivo assay may be used to determine the functional activity of pharmaceutical compositions described herein. In such assays, subjects may be exposed to protozoans and treated with a pharmaceutical composition of the invention. Infection may be assayed by protozoan load and/or survival of the experimental subjects. In addition, measurements of infection may be utilized to assess activity, including antibody titer, and symptoms as described herein below. These measurements can then be compared to corresponding measurements in control subjects. For example, test and control subjects may be inoculated with protozoan and serum samples may be drawn from the subjects after the final inoculation (for example every one or two weeks after inoculation). Test subjects also are administered a pharmaceutical composition of the invention and control subjects are not. Serum from the subjects can be analyzed for infection using known methods in the art as described herein below. Such assays may be used to compare levels of putative pharmaceutical composition to control levels of protozoan infection in a subject not administered the pharmaceutical composition as an indication that the putative pharmaceutical composition is effective to modulate protozoan infection.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Loss of SL Synthesis Leads to Hypersensitivity to Itraconazole and Ketoconazole in L. major Promastigotes

Anti-fungal drugs itraconazole (ITZ) and ketoconazole (KEZ) are potent inhibitors of the cytochrome P450-dependent lanosterol 14α-demethylase [Georgopapadakou, 1998; Georgopapadakou, et al 1996], a key enzyme in the synthesis of ergosterol. Both drugs have shown to possess anti-leishmanial activity against promastigotes and amastigotes [Hart, et al., 1989; Zakai, et al., 2000]. For L. major LV39 clone 5 wild type (WT) parasites, EC50s (concentrations of drugs required to inhibit growth by 50%) for ITZ and KEZ were around 1.1 μM and 3.1 μM, respectively (FIGS. 1A and 1D), similar to published values in other Leishmania species [Berman, et al., 1984; Beach, et al., 1988]. SL-null spt2⁻ parasites were extremely sensitive to ITZ and KEZ, as their EC50s (3.9 nM for ITZ and 10 nM for KEZ) were about 300 times lower than LV39 WT parasites (FIGS. 1A and 1D). This susceptibility is solely due to the lack of SLs because: 1) restoration of SL synthesis by episomal expression of the SPT2 gene shifted the EC50 back to WT level (FIG. 1A, spt2⁻/+SPT2); 2) WT parasites grown in the presence of 10 μM MYR, which was sufficient to shut down the synthesis of SLs [Zhang, et al., 2003], showed similar sensitivity to ITZ as spt2⁻ parasites (FIG. 1A, EC50 for WT+10 μM myriocin was about 4.6 nM); 3) recent evidence indicated that the primary role of SL metabolism in Leishmania promastigotes was to provide ethanolamine, which was essential for differentiation [Zhang, et al., 2006], yet supplementation of ethanolamine or choline has almost no effect on the sensitivity to ITZ (FIG. 1F) or KEZ (data not shown) in spt2⁻ parasites.

### Example 2

### Combinations of MYR and ITZ Inhibit the Growth of L. major Promastigotes Synergistically

The fact that MYR (10 μM) treatment drastically increased the susceptibility of LV39 WT parasites to ITZ (EC50 was reduced from 1.1 μM to 4.6 nM, FIG. 1A) prompted examination of whether ITZ could also work synergistically with MYR. Indeed, without ITZ, the EC50 for MYR in LV39 WT parasites was about 55 μM (very close to the solubility limit), whereas in the presence of 0.5 μM ITZ, it dropped to about 1.8 μM (FIG. 1B). Furthermore, combinations of these two drugs were tested and EC50s were determined as following: no ITZ/55 μM MYR, 0.92 nM ITZ/25 μM MYR, 4.6 nM ITZ/10 μM MYR, 131 nM ITZ/2.5 μM MYR, 250 nM ITZ/2.2 μM MYR, 500 nM ITZ/1.8 μM MYR, 750 nM ITZ/1.5 μM MYR, and 1.1 μM ITZ/no MYR. These results were plotted on a classical isobologram (FIG. 1C) and the fractional inhibitory concentration (FIC) was determined to be 0.06 (FIC<0.5 is considered synergistic, [Hallander, et al., 1982] which indicates a strong synergy between ITZ and MYR on the growth of L. major promastigotes [Hallander, et al., 1982]. This synergistic inhibition is not limited to the LV39 strain because very similar results were observed in another L. major strain, FV1 (MHOM/IL/80/Friedlin V1, data not shown). Interestingly, combinations of ITZ and MYR did not show much synergy against the in vitro growth of two pathogenic fungi, Cryptococcus neoformans (strain H99) and Candida albicans (strain CAI-4).

EPL-null mutant ads1− were challenged with several drugs listed in Table 1. In comparison to FV1 WT (the parental strain from which the ads1− mutant was generated), ads1− mutant was not only hypersensitive to ergosterol synthesis inhibitors like ITZ and KEZ (FIG. 1H and data not shown), but also to SL synthesis inhibitors like MYR (FIG. 1I). Together these data suggest the lipid triumvirate of ergosterol, SLs and EPLs ensures normal membrane function in Leishmania. Loss of one member of this triumvirate is not detrimental, but does lead to extreme vulnerability to further lipid perturbations.

Unlike ITZ or KEZ, other triazole or imidazole antifungals such as clotrimazole (CLT), miconazole, and fluconazole showed only modest selectivity against the SL-null spt2⁻ parasites (EC50s for these drugs in spt2⁻ were 2˜6 times lower in LV39 WT, FIG. 1E, Table 1). Similarly, inhibitors of other enzymes in the ergosterol synthesis pathway such as 3-(Biphenyl-4-yl)-3-hydroxyquinuclidine (BPQ-OH, inhibits squalene synthase), 22,26-azasterol (inhibits D24(25)-sterol methyltransferase), terbinafine (inhibits squalene epoxidase), and mevinolin (inhibits HMG-COA reductase), did not cause the extreme hypersensitivity in spt2⁻ parasites as seen with ITZ and KEZ (Table 1). Inhibitors of other classes of lipids including aureobasidin A (inhibitor of inositol phosphorylceramide synthase in fungi [Kuroda, et al., 1999; Zhong, et al., 2000]), miltefosine and eldefosine (both are lysophospholipid analogs and inhibit CTP: phosphocholine cytidyl transferase) also failed to show strong selectivity against spt2⁻ parasites (Table 1). In addition, membrane perturbation agents such as amphotericin B (which binds to ergosterol/sterol and interferes with membrane permeability) and cinnamycin (which binds to phosphatidyl-ethanolamine and induces cytolysis) inhibited WT and spt2⁻ parasites nearly equally well (Table 1 and data not show). Effects of terbinafine, miltefosine, edelfosine, and aureobasidin A were also tested using the EPL-null mutant ads1− along with FV1 WT parasites and results showed very similar EC50s (data not shown).

### Example 3

### ITZ Inhibits Targets Beyond Lanosterol 14a-demethylase in Leishmania promastigotes

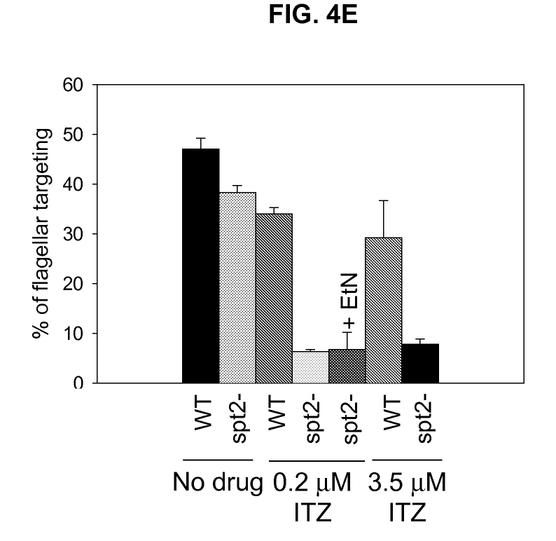

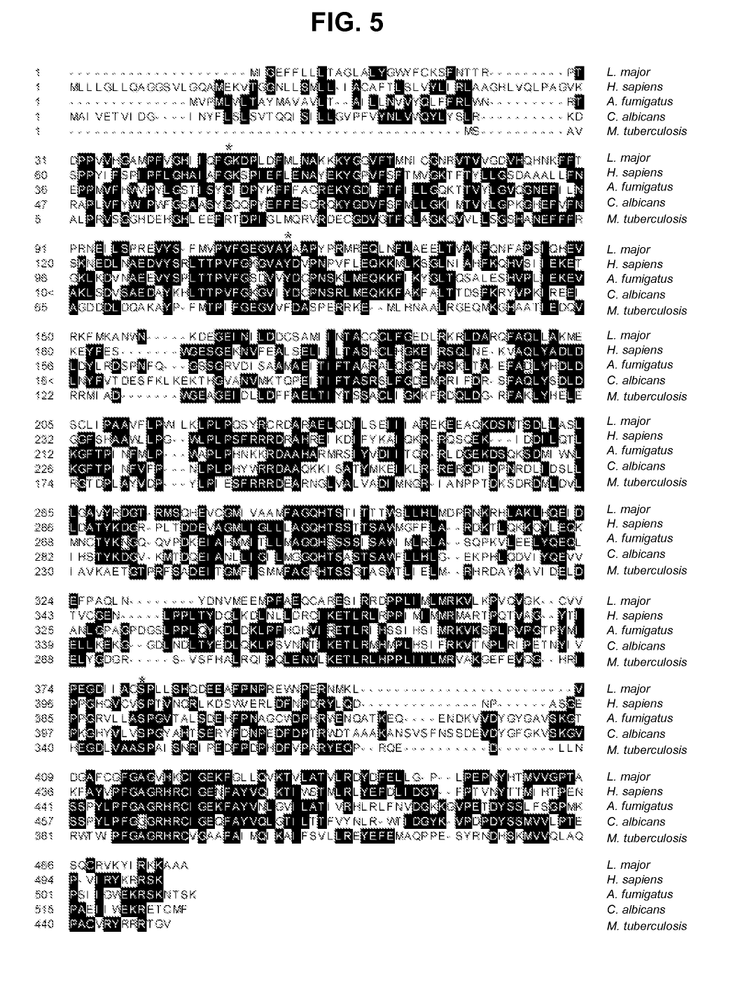

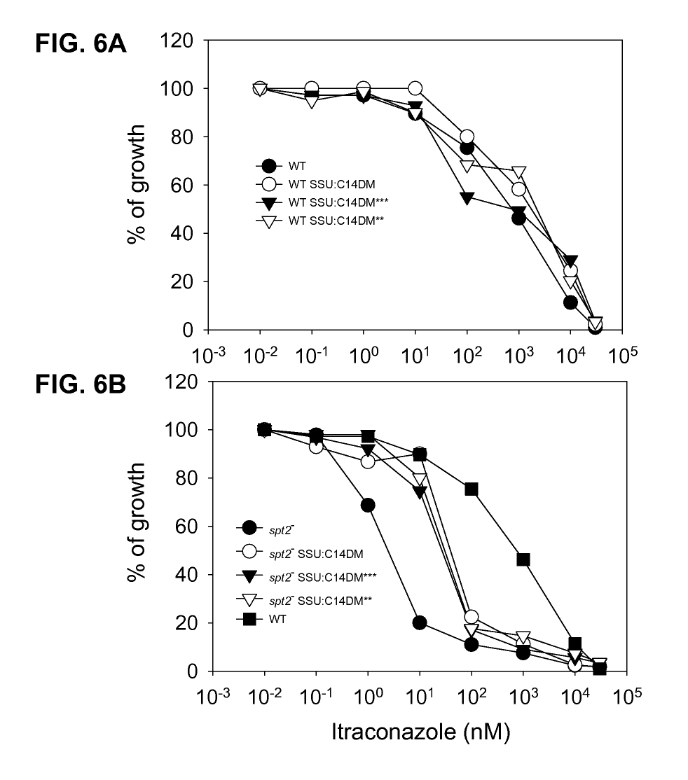

To test whether ITZ inhibits the same target in L. major as in fungi, a putative lanosterol 14α-demethylase (LmC14DM) gene was identified in L. major, which was ˜48% identical to the C14DM in A. fumigates at the amino acid level (FIG. 5). This gene was then cloned and integrated into the small ribosomal unit site of both WT (WT SSU::C14DM) and spt2⁻ (spt2⁻ SSU:C14DM) parasites to achieve a high level of expression [Robinson, et al., 2003] as described in the Materials and Methods below. Both control and transfected parasites were tested for sensitivity to ITZ, and results were shown in FIG. 1G. Overexpression of LmC14DM did buffer the effect of ITZ on sterol composition at low nanomolar range (Table 2). However, such overexpression only conferred ˜ten-fold increase in EC50 in spt2−, and had very little effect on WT parasites (FIG. 1G), suggesting LmC14DM is not the only target of ITZ in L. major. In addition, mutations were introduced at several conserved amino acids in LmC14DM (G49R, Y115H, and S382F, see FIG. 5, asterisks) based on research on fungal C14DM [Kakeya, et al., 2000; Diaz-Guerra, et al., 2003; Sanglard, et al., 1998]. Although the equivalent mutations confer substantial resistance to azole drugs in fungal enzymes [Kakeya, et al., 2000; Diaz-Guerra, et al., 2003; Sanglard, et al., 1998], these modified LmC14DM genes had very similar effects as the unmodified gene when overexpressed in Leishmania parasites (FIGS. 6A and 6B). Together, these results suggest LmC14DM is not the only target of ITZ in L. major.

TABLE 2
Effects of ITZ and CLT on the composition of free sterols in WT and
spt2⁻ promastigotes.

| | | | 5- | | | | | |
|---|---|---|---|---|---|---|---|---|
| | | | dehydro- | | | 14-methyl- | | |
| | Growth | Ergosterol | episterol | Episterol | Cholesterol | fecosterol | Lanosterol | Total |
| Sample | (%) | (%) | (%) | (%) | (%) | (%) | (%) | sterols/cell |
| WT control | 100 | 33.0 | 55.5 | 6.4 | 3.6 | 1.5 | ND | 2.6 × 10⁸ |
| WT + 0.2 μM | 76.9 | 4.4 | 63.7 | 17.5 | 4.5 | 9.9 | ND | 3.3 × 10⁸ |
| CLT | | | | | | | | |
| WT + 0.5 μM | 78.7 | 1.9 | 42.0 | 5.1 | 3.9 | 47.1 | ND | 2.2 × 10⁸ |
| CLT | | | | | | | | |
| WT + 1 nM | 91.5 | 24.3 | 9.6 | 0.9 | 2.9 | 62.3 | ND | 2.0 × 10⁸ |
| ITZ | | | | | | | | |
| WT + 5 nM | 81.6 | 2.9 | 1.7 | ND | 3.2 | 92.2 | ND | 3.3 × 10⁸ |
| ITZ | | | | | | | | |
| spt2⁻ control | 100 | 31.7 | 42.1 | 6.1 | 18.2 | 1.4 | ND | 3.6 × 10⁸ |
| spt2⁻ + 0.2 μM | 93.8 | 3.5 | 44.1 | 10.7 | 20.2 | 21.5 | ND | 3.5 × 10⁸ |
| CLT | | | | | | | | |
| spt2⁻ + 0.5 μM | 62.8 | 1.2 | 23.9 | 3.5 | 21.1 | 50.3 | ND | 3.1 × 10⁸ |
| CLT | | | | | | | | |
| spt2⁻ + 1 nM | 64.6 | 13.2 | 3.7 | 1.6 | 22.0 | 59.4 | 0.1 | 4.0 × 10⁸ |
| ITZ | | | | | | | | |
| spt2⁻ + 5 nM | 29.4 | 4.0 | 2.1 | 0.4 | 17.1 | 75.2 | 1.2 | 4.4 × 10⁸ |
| ITZ | | | | | | | | |

Log phase L. major LV39 WT and spt2⁻ promastigotes were grown in the presence or absence of ITZ or CLT. Culture densities were determined 48 hours later and total lipids were extracted and the composition of free sterols was determined by GC/MS as described in Materials and Methods. ND: not detectable. Each experiment was repeated at least 3 times and results from one representative set were shown.

### Example 4

### Ether Phospholipid-Null Mutant is Hypersensitive to ITZ and MYR

The plasma membrane of Leishmania parasites is rich in ergosterol, ether phospholipids (plasmalogen PE and alkyl-acyl-PI), and IPC. One explanation to why genetic or chemical depletion of IPC leads to extreme hypersensitivity to ITZ/KEZ is that parasites need at least two out of these three classes of lipids to maintain normal membrane functions. This hypothesis was challenged in an ads1 mutant, which is null in the synthesis of all ether phospholipids but maintains normal levels of other phospholipids and SLs [Zufferey, et al., 2003], with several drugs listed in Table 1. In comparison to FV1 WT (the reference strain from which the ads1 mutant was generated), ads1− mutant was hypersensitive to not only ITZ and KEZ (FIG. 1H and data not shown), but also to MYR (FIG. 1I). However, other inhibitors that were tested including miltefosine, edelfosine, and terbinafine showed little or no selectivity against ads1⁻ (data not shown). Together these data suggest the combination of ergosterol, SLs and ether phospholipid ensures normal membrane function in Leishmania, and loss of one member of this lipid triumvirate renders parasites vulnerable to perturbations of the remaining two.

### Example 5

### ITZ Treatments Result in Depletion of Endogenous Ergosterol Species and Accumulation of 14-methylfecosterol

Azole drugs such as fluconazole, ITZ, KEZ, and CLT cause accumulation of 14-methyl sterols (substrates of lanosterol 14α-demethylase; C14DM) and depletion of ergosterol in fungi [Vanden Bossche, et al.; 1998; Martin, 1999; Bailey, et al., 1990]. A similar mode of action for ITZ and KEZ has been reported in Leishmania spp [Hart, et al., 1989; Goad, et al., 1985; Berman, et al., 1984]. The extreme sensitivity of spt2⁻ parasites to ITZ/KEZ raises the question of whether ITZ/KEZ can inhibit LmC14DM at low nanomolar concentrations. The fact that only ITZ and KEZ (but not other ergosterol synthesis inhibitors) work synergistically with MYR suggests: (1) these drugs may not inhibit C14DM in L. major, (2) they may have additional targets besides C14DM. To answer these questions, WT and spt2⁻ parasites were grown in various concentrations of ITZ or CLT (0.01 nM to 1 μM), and effects on sterol lipids were analyzed 48 hours later by gas chromatography/mass spectrophotometry (GC/MS) and summarized in Table 2. Without drug treatment, the composition of detectable free sterols in WT parasites is: 55.5% of 5-dehydroepisterol, 33.0% of ergosterol, 6.4% of episterol, 3.6% of cholesterol, and 1.5% of 14-methylfecosterol; in spt2⁻ parasites is: 42.1% of 5-dehydroepisterol, 31.7% of ergosterol, 6.1% of episterol, 18.2% of cholesterol, and 1.4% of 14-methylfecosterol (Table 2). Clearly, in the absence of drugs, 5-dehydroepisterol and ergosterol made up the majority of free sterols in WT and spt2⁻ parasites, consistent with previous reports on other Leishmania spp [Berman, et al., 1984; Beach, et al., 1988. The relative abundance of cholesterol, which was not synthesized by Leishmania spp and salvaged from the medium [Goad, et al., 1985], was 4˜5 times higher in spt2⁻ (18.2%) than in WT (3.6%) parasites and was not affected by ITZ or CLT treatments (Table 2). Importantly, 1-5 nM of ITZ was sufficient to deplete the majority of endogenous ergosterol lipids (5-dehydroepisterol, ergosterol, and episterol) in both WT and spt2⁻ parasites, which were largely replaced by 14-methylfecosterol (Table 2), yet such ITZ exposure had much more pronounced anti-proliferative effect in spt2⁻ than in WT parasites (Table 2). While KEZ has very similar effects as ITZ (data not shown), CLT, on the other hand, was quite different in several aspects. First, unlike ITZ, it had very little effect on growth or sterol composition at concentrations less than 100 nM (data not shown). Second, at 0.2˜1 μM, CLT selectively depleted ergosterol before 5-dehydroepisterol in both WT and spt2-parasites (Table 2). Despite changes in sterol composition, total amounts of free sterols did not show huge variations after drug exposure (2˜5×10⁸/cell, Table 2). Together, these results suggest: (1) the ergosterol depletion by ITZ/KEZ is sufficient to cause severe growth retardation only in the absence of SLs; (2) ITZ/KEZ probably have additional targets besides C14DM in WT Leishmania.

### Example 6

### ITZ Disrupts Detergent Resistant Membrane Fractions in L. major Promastigotes

Sterols play important roles in the organization of membrane lipids and stabilization of ordered domains such as “rafts” [Mouritsen, et al., 2004]. Given the potent effect of ITZ on sterol composition, it is of great interest to determine whether such changes would affect the integrity or stability of organized membrane domains.

Detergent resistant membrane fractions (DRMs) were prepared using Triton X-100 from L. major promastigotes as previously described [Zhang, et al., 2003. Such DRMs are usually enriched for raft-associated materials and have been used to characterize rafts [London, et al., 2000; Brown, et al., 1998]. A typical marker for a raft in Leishmania parasites is GP63, a GPI-anchored metalloprotease, which can be detected from DRM samples by western-blot [Zhang, et al., 2003].

As shown in FIG. 2, without ITZ, about half of GP63 was insoluble after Triton X-100 extraction (54% for WT and 46% for spt2⁻ parasites) at 4° C., whereas at 37° C. the majority of GP63 was soluble, a typical feature for raft proteins [Schuck, et al., 2003]. At concentrations higher than 1 nM, ITZ caused significant reduction of insoluble GP63 at 4° C. (FIG. 2B) in spt2⁻ parasites, suggesting rafts were disturbed. However, in WT parasites, ITZ did not cause the same kind of disruption even at 100 nM (FIG. 2A), suggesting: 1) the loss of SLs changes the membrane physiology and renders the rafts in spt2− parasites more sensitive to perturbations of sterol synthesis; 2) 14-methylfecosterol, the accumulated product after ITZ treatment, cannot fully substitute the function of endogenous sterols (ergosterol, 5-dehydroepisterol, and episterol).

### Example 7

### Growth Inhibition Induced by ITZ and CLT is Associated with Disruption of DRM

Here WT and spt2⁻ parasites were grown in various concentrations of ITZ (0.001 nM to 1 μM) or CLT (10 nM to 5 μM). Effects of ITZ and CLT on growth, sterol composition and DRM were analyzed after 48 hours. Basically, culture densities were determined using a particle counter and drugs' effects on growth were assessed in comparison to control cells (no drug); sterol lipids were extracted and analyzed by GC/MS and the percentage of 14-methylfecosterol (among all detectable sterol lipids) was used to reflect changes in sterol composition (see Table 2). DRMs were also isolated and the percentage of GP63 that were insoluble at 4° C. was used as an indicator for the integrity of DRM (see FIG. 2). Two to four independent experiments were performed and the averaged results were plotted in FIG. 3.

Consistent with the data described earlier, 1˜10 nM of ITZ were sufficient to shut down LmC14DM and lead to a huge accumulation of 14-methylfecosterol (from 1˜2% to 80˜92%, FIG. 3B) and depletion of endogenous sterols (from 85˜95% to 4˜6%, FIG. 3B) in both WT and spt2⁻ parasites. Yet at such concentrations of ITZ, WT parasites were able to grow fairly well (75˜95% of control, FIG. 3A) and maintain the integrity of their DRMs (40˜55% of GP63 in DRM versus 50˜55% in control cells, FIG. 3C). In contrast, 1˜10 nM of ITZ significantly reduced the proliferation of spt2⁻ parasites (28˜67% of control, FIG. 3A) and destabilized their DRMs (24˜28% of GP63 in DRM versus 45˜50% in control cells, FIG. 3C). These results again suggest that in the absence of SLs, spt2⁻ parasites depend more on endogenous sterols (ergosterol, 5-dehydroepisterol, and episterol) to maintain DRMs. At concentrations more than 100 nM, ITZ was able to significantly inhibit the growth and DRM stability in WT parasites (FIGS. 3A and 3C), suggesting there are additional target(s) besides LmC14DM since 10 nM of ITZ was sufficient to completely alter the sterol composition yet had only marginal effect on growth (FIG. 3B).

Response of parasites to CLT, however, was quite different. First, a much higher concentration of CLT (>100 nM) was needed to achieve any noticeable effects on growth, sterol composition, or DRMs (FIG. 3D-3F), suggesting CLT had a lower affinity for LmC14DM than ITZ did. Second, sterol compositions in WT and spt2⁻ parasites were almost equally sensitive to CLT (FIG. 3E), and although the DRM and growth rate in spt2⁻ parasites were slightly more sensitive to CLT than in WT parasites, the difference was not nearly as pronounced as with ITZ (FIGS. 3A, 3C, 3D and 3F). Overall, these data reveal a clear correlation between the disruption of DRMs and growth inhibition in L. major promastigotes after exposure to ITZ or CLT.

### Example 8

### ITZ Interferes with the Flagellar Localization of FcaBP in spt2⁻ Promastigotes

In many organisms, a subset of membrane proteins are targeted to lipid rafts and localized to specific sites on the plasma membrane, which is crucial for their functions in signaling [Proszynski, et al., 2006; Grossmann, et al., 2006]. The ability of ITZ to disrupt DRMs raises the question of whether ergosterol-related lipids are required for the correct localization of raft proteins in Leishmania spp. In Trypanosoma cruzi, a flagellar calcium-binding protein (FcaBP) is known to associate with the flagellar membrane, which is enriched in raft proteins (personal communications from Dr. David Engman from Northwestern University), via N-terminal myristoylation and palmitoylation in a calcium-modulated, conformation-dependent manner [Buchanan, et al., 2005; Godsel, et al., 1999]. Here we introduced a HA-tagged FcaBP (FcaBP-HA) into L. major promastigotes and used it to probe the role of sterols and SLs in the localization of raft proteins.

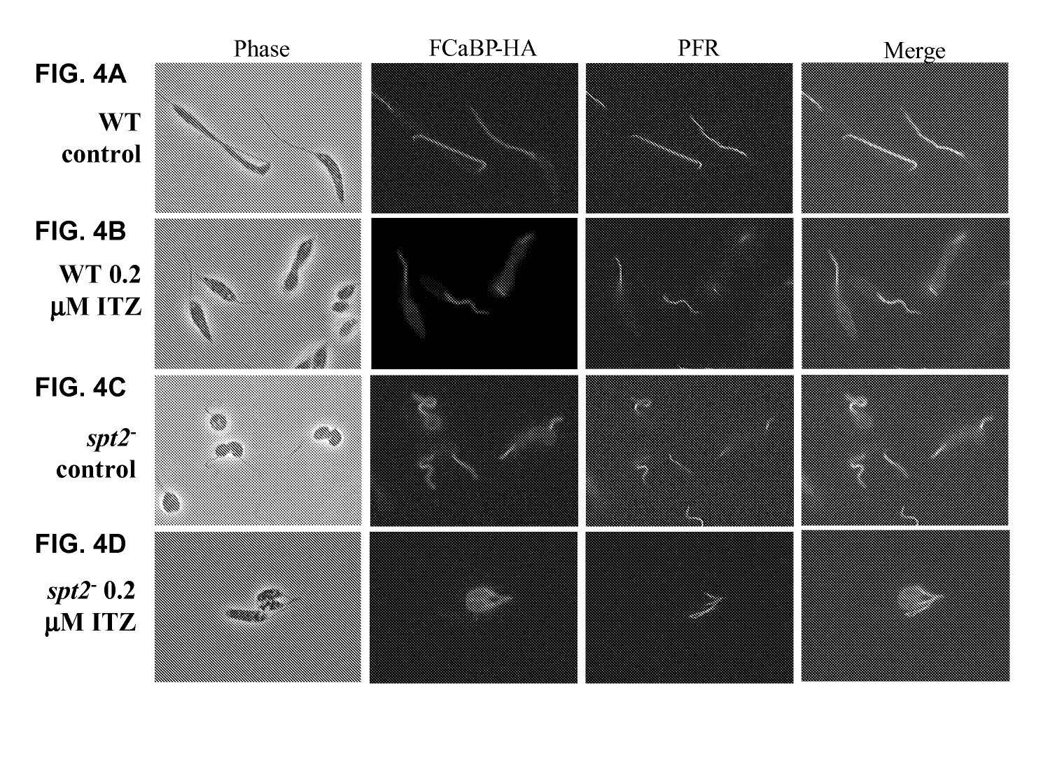

In the absence of sterol-depleting agents like ITZ, FcaBP-HA was associated with DRM rafts (data not shown) and primarily localized to the flagella in both WT and spt2⁻ parasites, as shown by the co-staining with an anti-T. brucei paraflagellar rod (PFR) protein antibody (FIGS. 4A, 4C and 4E). After exposure to ITZ (200 nM and 3.5 μM) for 20 hours, which was sufficient to cause disorganization of DRMs (data not shown), WT parasites could still target FcaBP-HA to flagella, although at slightly lower efficiencies (FIGS. 4B and 4E). In contrast, the same ITZ treatments resulted in significant mislocalization of FcaBP-HA in spt2⁻ parasites (FIGS. 4D and 4E), as FcaBP-HA was distributed not only to the flagellar membrane, but also the plasma membrane around the cell body (FIG. 4D). As expected, addition of ethanolamine did not have any effect on the localization of FcaBP-HA (FIG. 4E), indicating this is a SL-dependent phenotype. This mislocalization of FcaBP-HA was not a secondary effect of growth inhibition because parasites treated with other drugs like terbinafine (using concentration 5˜10 times of EC50 for 20 hours) did not show such defect (data not shown). These results suggest although ergosterols and DRMs are not absolutely essential for the correct targeting of FcaBP-HA in WT parasites, loss of SLs renders spt2⁻ parasites more dependent on ergosterols to maintain the proper trafficking of certain membrane proteins. Importantly, not all membrane proteins were mislocalizd in spt2⁻ parasites treated with ITZ, e.g. GP63 and hydrophilic acylated surface protein b (HASPb, [Denny, et al., 2000]) were not altered under such conditions (data not shown).

### Example 9

### Screening for Novel ‘Synergy’ Drugs

Wild-type Leishmania major LV39 clone 5 promastigotes were grown in a microtiter plate format. Following inoculation, parasites were allowed to grow until late logarithmic phase. At that time, parasite growth was estimated following lysis and quantitation of cellular ATP levels using a luciferase based assay described previously by Mackey et al 2006, previously incorporated herein by reference. Parasites were inoculated without any drug, singly with the two prototypic synergistic drugs myriocin or itraconazole, or both drugs, at varying concentrations. As expected from previous experiments performed in T25 culture flasks, a high degree of synergy was seen.

Parasites were tested against 320 compounds (arising from the first 4 plates in their Spectrum Drug collection, each at a concentration of 1 μM. For each test compound, three tests of parasite growth were performed; one without further addition (0 drug control), one in the presence of myriocin (40 μM), and one in the presence of itraconazole alone (25 nM). The format, statistical analysis and various controls were as described (Mackey et al 2006). From the data each drug was classified as follows:

| Class | | Number of compounds |
|---|---|---|
| a) | growth in all three conditions - inactive | 291 |
| b) | strong inhibition (>95%) in all | 10 |
| | three conditions | |
| c) | strong growth inhibition ONLY | 2 |
| | in presence of myriocin | |
| d) | strong growth inhibition ONLY | 3 |
| | in the presence of itraconazole | |
| e) | other/weak synergy | 14 |

### Compounds falling into classes c) and d) represent candidate ‘synergistic’ partners for myriocin and itraconazole respectively.

Two compounds that were tentatively identified as synergistic with myrioscin (class c) were salinomycin and isoliquiritigenin. The three compounds that identified as synergistic with itraconazole (class d) were chlorzoxazone, cinoxacin, and penicillic acid. Controls using myriocin and itraconazole alone or in combination performed as expected. Synergy of cinoxacin was not confirmed, but for penicillic acid and chlorzoxaone synergy was confirmed, although the effect was modest (˜2 fold).

In conclusion, this pilot study demonstrates that the invention provides a high throughput method for screening the mutant strain of Leishmania major; confirms previously identified synergistic partners; shows that this method can yield ‘hits’ from libraries of experimental test compounds; hits were validated at a reasonable frequency (⅖); hits obtained were only weakly synergistic; and efficacy of the screen establishes that strongly active compounds will be recovered if present.

### Materials and Methods for Examples 1-9

### Cell Culture and Growth Inhibition Assays

Wild type (WT) L. major LV39 clone 5 (Rho/SU/59/P), FV1 (MOHM/IL/80/Friedlin virulent clone 1), spt2⁻ (Δspt2::HYG/Dspt2::PAC), ads1⁻ (Δads1::HYG/Dads1::SAT), and spt2⁻/+SPT2 (Δspt2::HYG/Dspt2::PAC/+pXG-SPT2) cells were grown in M199 with 10% FBS and supplements as described [Zhang, et al., 2003; Kapler, et al., 1990]. For growth inhibition assays, promastigotes were inoculated at 1.0×10⁵ cells/ml (in T25 flasks, 5 ml of medium per flask) in various concentrations of drugs. All cultures contained equivalent amount of DMSO or methanol (0.1% or less, from drug stocks). After 48 or 96 (for ads1⁻ only) hours, culture densities were determined using a Coulter counter (Z1, Beckman) and effects of drugs on growth were assessed in relative to cultures grown in the absence of drugs. For a particular drug, the EC50 was determined by calculating the concentration needed to inhibit growth by 50% comparing to control culture (no drug).

Effects of ITZ and MYR on C. neoformans and C. albicans were determined in a similar fashion except fungal cells were grown in YPD medium in a shaker (300 rpm, 30° C.) and culture densities were measured by OD600 nm reading.

### Synergy Calculations

In these studies, synergy is defined as an effect produced by a combination of inhibitors that is greater than the sum of the effects produced by the inhibitors alone [Hallander, et al., 1982]. A classical isobologram was constructed by plotting EC50s of drugs either alone or in combination. Fractional inhibitory concentration (FIC) was calculated as previously described: FIC=EC50_{XY}/EC50_{X}+EC50_{YX}/EC50_{Y}, where EC50_{X} is the EC50 value for drug X acting alone, and EC50_{XY} is the EC50 of the same drug in the presence of a sub-optimal concentration of drug Y. Similarly, EC50_{Y} is the EC50 value for drug Y acting alone, and EC50_{YX} is the EC50 of the same drug in the presence of a sub-optimal concentration of drug X. If the value of the FIC is 0.5, a synergic effect is diagnosed, for 0.5<FIC 1 the effects are simply additive and for FIC>1.0 the combined effects are considered antagonistic [Hallander, et al., 1982].

### Molecular Constructs and Leishmania Transfections

The putative L. major lanosterol 14α-demethylase gene (systematic ID: LmjF11.1100) was PCR amplified from the genomic DNA of LV39 WT using oligos #2259 (5′-TAC TAG AGA TCT CCA CCA TGA TCG GCG AGT TCT TCC T-3′) (SEQ ID NO: 6) and #2260 (5′-TAC TAG AGA TCT CTA AGCAGC CGC CTT CTT CC-3′) (SEQ ID NO: 7). The resulting 1.45 Kb DNA fragment (LmC14DM) was digested with Bgl II and cloned into expression vector pIRPhleo [Zhang, et al., 2004] to make pIRPhleo-LmC14DM (B5389). This construct was then linearized with Swal and transfected into LV39 WT and spt2⁻ promastigotes as previously described [Robinson, et al., 2003]. Colonies resistant to phleomycin (15 mg/ml) were selected as WT SSU:C14DM and spt2⁻ SSU:C14DM.

To generate the HA-tagged flagellar calcium binding protein from T. cruzi (FcaBP-HA), PCR reactions were performed using oligos #2321 (5′-AGGGCAAGATCTCCACCATGGGTGCTTGTGGGTCGA-3′) (SEQ ID NO: 8) and #2322 (5′-GTGGAGATCTTTACGCGTAGTCCGGGACGTCGTACGGGTAG CCCGCGCTCTCCGGCACGT-3′) (SEQ ID NO: 9) and the plasmid DNA of pTEX-FcaBP-9E10 (kindly provided by Dr. David Engman, Department of Pathology, Northwestern University Medical School) as template. The resulting DNA fragment (˜660 bp) was digested with Bgl II and cloned into expression vector pXGPhleo to make pXGPhleo-FcaBP-HA (B5457), which was introduced into LV39 WT and spt2⁻ promastigotes as described above.

### Drugs, Inhibitors and Antibodies

Itraconazole, ketoconazole, clotrimazole, miconazole, fluconazole, and terbinafine were purchased from LKT Laboratories, Inc (ST Paul, Minn.). Myriocin, mevinolin, amphotericin B, edelfosine (1-O-octadecyl-2-O-methyl-rac-glycero-3-phosphocholine), and cinnamycin were purchased from Sigma-Aldrich Co (St. Louis, Mo.). Aureobasidin A was purchased from PanVera Corp (Madison, Wis.). Miltefosine (hexadecyl-phosphocholine) was purchased from EMD Biosciences, Inc (San Diego, Calif.). 3-(Biphenyl-4-yl)-3-hydroxyquinuclidine (BPQ-OH) and 22,26-azasterol were kindly provided by Dr. Julio Urbina (Laboratorio de Quimica Biologica, Centri de Bioquimica y Biofisca, Instituto Venezolano de Investigaciones Cientificas, Caracas, 1020, Venezuela). Stock solutions were made in DMSO or methanol and stored at −20° C.

Rabbit anti-HA antibody was purchased from Sigma-Aldrich Co (St. Louis, Mo.). Monoclonal antibody (L8C4) against T. brucei paraflagellar rod (PFR) protein was a generous gift from Dr. Keith Gull (Sir William Dunn School of Pathology, University of Oxford, Oxford, United Kingdom).

### Lipid Extraction and Analysis by Gas Chromatography/Mass Spectrophotometry (GC/MS)

Total lipids were extracted using the Folch's method [Folch, et al., 1957]. Briefly, promastigotes were harvested by centrifugation and washed once with PBS. Pellets were resuspended in chloroform:methanol (2:1) at 1.0×10⁸ cells/ml with the addition of cholesta-3,5-diene (Sigma-Aldrich, FW 368.7) at 0.5 mg/10⁸ cells (6.6×10⁷/cell). After vortexing for 30 s, cell debris was removed by centrifugation (1000 g for 10 min) and samples were washed with 0.2 volume of 0.9% NaCl. After centrifugation, supernatants were removed and the organic phase solutions were dried under a stream of nitrogen. Samples were then dissolved in chloroform:methanol (1:2) at 1.0×10⁹ cells/ml.

Electron impact gas chromatography/mass spectrometry (GC/MS) analyses of sterol lipids were performed on a Finnigan (San Jose, Calif.) SSQ-7000 single-stage quadrupole mass spectrometer with a Varian (Walnut Creek, Calif.) 3400 GC, which is controlled by Finnigan ICIS software operated on a DEC alpha station. The extracts in solution (1 mL) were injected in a splitless mode and analyzed by GC on a Restek (Bellefonte, Pa.) RTX-5 column (15 m, 0.33 mm id, 1 mm film thickness). The initial temperature of GC was set at 80° C. for 1 min, increased to 220° C. at a rate of 50° C./min, and then raised to a final temperature of 280° C. at a rate of 10° C./min. The temperatures of the injector, transfer line of the GC column, and of the ion-source were set at 280° C., 280° C., and 240° C., respectively. The full scan mass spectra (50 to 500 Dalton) were acquired at a rate of 1 scan/0.25 Sec.

### Western Blot with Anti-GP63 Antibody

Detergent resistant membrane fractions (DRMs) were isolated and a western blot with a monoclonal antibody again Leishmania GP63 #235 was performed as previously described [Zhang, et al., 2003]. Percentages of GP63 in soluble and insoluble fraction after extractions at 4° C. were determined using a Fuji FLA5000 phosphoimager.

### Immunofluorescent Microscopy

LV 39 WT and spt2⁻ promastigotes transfected with pXGPhleo-FCaBP-HA were attached to polylysine coated cover slips, followed by fixation with 3.7% formaldehyde in PBS. Cells were stained with a rabbit anti-HA:antibody (1:200) and a monoclonal antibody (L8C4) against T. brucei paraflagellar rod (PFR) protein (1:10), followed by secondary antibodies (anti-rabbit IgG-Texas Red and anti-mouse IgG-FITC). Images were obtained using an Olympus AX70 microscope. Percentages of cells showing flagellar localization of FcaBP-HA were determined after examinations of 150-200 randomly selected cells.

### Mutagenesis of LmC14DM Gene

To introduce the desired mutations (G49R, Y115H, and S382F) in LmC14DM, we used a QuikChange MultiSite-Directed Mutagenesis Kit (Stratagene, La Jolla, Calif.) and followed the recommended protocol. Three mutagenic primers were used: 5′-ACATCATCCAGTTCC-GCAAGGATCCGCTGG-3′ (SEQ ID NO: 10) (#2456, to introduce G49R); 5′-GAGGGCGTCGCCCA-CGCCGCGCCATACCCG-3′ (SEQ ID NO: 11) (#2457, to introduce Y115H); 5′-CATCATCGCCTGCTT-CCCGCTCCTCTCGC-3′ (SEQ ID NO: 12) (#2458, to introduce S382F). Plasmid DNA of pIRPhleo-LmC14DM (B5389) was used as a template. Two constructs carrying mutated LmC14DM genes were obtained and confirmed by DNA sequencing: pIRPhleo-LmC14DM*** (B5714) which contains three mutations (G49R, Y115H, and S382F) and pIRPhleo-LmC14DM** (B5715) which contains two mutations (Y115H, and S382F). Both plasmids were linearized with Swal and integrated into the small ribosomal site of LV39 WT and spt2⁻ promastigotes as previously described [Robinson, et al., 2003]. Colonies resistant to phleomycin (15 mg/ml) were selected as WT SSU:C14DM***, WT SSU:C14DM**, spt2⁻ SSU:C14DM*** and spt2⁻ SSU:C14DM**.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

### REFERENCES

The following references, to the extent that they provide exemplary procedural or other details supplementary to those set forth herein, are specifically incorporated herein by reference.
1. Croft, S. L. and G. H. Coombs, Leishmaniasis—current chemotherapy and recent advances in the search for novel drugs. Trends Parasitol, 2003. 19(11): p. 502-8.
2. Berman, J. D., Chemotherapy for leishmaniasis: biochemical mechanisms, clinical efficacy, and future strategies. Rev Infect Dis, 1988. 10(3): p. 560-86.
3. Davidson, R. N., et al., Liposomal amphotericin B in drug-resistant visceral leishmaniasis. Lancet, 1991. 337(8749): p. 1061-2.
4. Schmidt-Ott, R., et al., Topical treatment with hexadecylphosphocholine (Miltex) efficiently reduces parasite burden in experimental cutaneous leishmaniasis. Trans R Soc Trop Med Hyg, 1999. 93(1): p. 85-90.
5. Urbina, J. A., Lipid biosynthesis pathways as chemotherapeutic targets in kinetoplastid parasites. Parasitology, 1997. 114 Suppl: p. S91-9.
6. Georgopapadakou, N. H., Antifungals: mechanism of action and resistance, established and novel drugs. Curr Opin Microbiol, 1998. 1(5): p. 547-57.
7. Georgopapadakou, N. H. and T. J. Walsh, Antifungal agents: chemotherapeutic targets and immunologic strategies. Antimicrob Agents Chemother, 1996. 40(2): p. 279-91.
8. Holz, G. G., Lipids of leishmaniasis. In Leishmaniasis (Human Prasitic Diseases, Chang, K. P. and Bray, R. S. Eds). 1985.1: p. 79-92.
9. Kirkpatrick, W. R., et al., Combination therapy with terbinafine and amphotericin B in a rabbit model of experimental invasive aspergillosis. Antimicrob Agents Chemother, 2005. 49(11): p. 4751-3.
10. Perez, A., Terbinafine: broad new spectrum of indications in several subcutaneous and systemic and parasitic diseases. Mycoses, 1999. 42 Suppl 2: p. 111-4.
11. Vanden Bossche, H., et al., Antifungal drug resistance in pathogenic fungi. Med Mycol, 1998. 36 Suppl 1: p. 119-28.
12. Martin, M. V., The use of fluconazole and itraconazole in the treatment of Candida albicans infections: a review. J Antimicrob Chemother, 1999. 44(4): p. 429-37.
13. Bailey, E. M., D. J. Krakovsky, and M. J. Rybak, The triazole antifungal agents: a review of itraconazole and fluconazole. Pharmacotherapy, 1990. 10(2): p. 146-53.
14. Hart, D. T., et al., Perturbation of sterol biosynthesis by itraconazole and ketoconazole in Leishmania mexicana mexicana infected macrophages. Mol Biochem Parasitol, 1989. 33(2): p. 123-34.
15. Goad, L. J., G. G. Holz, Jr., and D. H. Beach, Sterols of ketoconazole-inhibited Leishmania mexicana mexicana promastigotes. Mol Biochem Parasitol, 1985. 15(3): p. 257-79.
16. Kaneshiro, E. S., K. Jayasimhulu, and R. L. Lester, Characterization of inositol lipids from Leishmania donovani promastigotes: identification of an inositol sphingophospholipid. J Lipid Res, 1986. 27(12): p. 1294-303.
17. van der Rest, M. E., et al., The plasma membrane of Saccharomyces cerevisiae: structure, function, and biogenesis. Microbiol. Rev, 1995. 59(2): p. 304-22.
18. Zufferey, R., et al., Ether phospholipids and glycosylinositolphospholipids are not required for amastigote virulence or for inhibition of macrophage activation by Leishmania major. J Biol Chem, 2003. 278(45): p. 44708-18.
19. Brown, D. A. and E. London, Structure and function of sphingolipid- and cholesterol-rich membrane rafts. J Biol Chem, 2000. 275(23): p. 17221-4.
20. London, E. and D. A. Brown, Insolubility of lipids in triton X-100: physical origin and relationship to sphingolipid/cholesterol membrane domains (rafts). Biochim Biophys Acta, 2000. 1508(1-2): p. 182-95.
21. Mattjus, P. and J. P. Slotte, Does cholesterol discriminate between sphingomyelin and phosphatidylcholine in mixed monolayers containing both phospholipids? Chem Phys Lipids, 1996. 81(1): p. 69-80.
22. Ohvo-Rekila, H., et al., Cholesterol interactions with phospholipids in membranes. Prog Lipid Res, 2002. 41(1): p. 66-97.
23. Pike, L. J., et al., Lipid rafts are enriched in arachidonic acid and plasmenylethanolamine and their composition is independent of caveolin-1 expression: a quantitative electrospray ionization/mass spectrometric analysis. Biochemistry, 2002. 41(6): p. 2075-88.
24. Zhang, K., et al., Sphingolipids are essential for differentiation but not growth in Leishmania. Embo J, 2003. 22(22): p. 6016-26.
25. Denny, P. W., et al., Sphingolipid-free Leishmania are defective in membrane trafficking, differentiation and infectivity. Mol Microbiol, 2004. 52(2): p. 313-27.
26. Pinto, W. J., et al., Sphingolipid long-chain-base auxotrophs of Saccharomyces cerevisiae: genetics, physiology, and a method for their selection. J Bacteriol, 1992. 174(8): p. 2565-74.
27. Hanada, K., et al., Specificity of inhibitors of serine palmitoyltransferase (SPT), a key enzyme in sphingolipid biosynthesis, in intact cells. A novel evaluation system using an SPT-defective mammalian cell mutant. Biochem Pharmacol, 2000. 59(10): p. 1211-6.
28. Zhang K., et al., Redirection of sphingolipid metabolism toward de novo synthesis of ethanolamine in Leishmania. EMBO J. 2007 Feb. 21; 26(4):1094-104.
29. Zakai, H. A. and S. K. Zimmo, Effects of itraconazole and terbinafine on Leishmania major lesions in BALB/c mice. Ann Trop Med Parasitol, 2000. 94(8): p. 787-91.
30. Berman, J. D., G. G. Holz, Jr., and D. H. Beach, Effects of ketoconazole on growth and sterol biosynthesis of Leishmania mexicana promastigotes in culture. Mol Biochem Parasitol, 1984. 12(1): p. 1-13.
31. Beach, D. H., L. J. Goad, and G. G. Holz, Jr., Effects of antimycotic azoles on growth and sterol biosynthesis of Leishmania promastigotes. Mol Biochem Parasitol, 1988. 31(2): p. 149-62.
32. Hallander, H. O., et al., Synergism between aminoglycosides and cephalosporins with antipseudomonal activity: interaction index and killing curve method. Antimicrob Agents Chemother, 1982. 22(5): p. 743-52.
33. Kuroda, M., et al., An aureobasidin A resistance gene isolated from Aspergillus is a homolog of yeast AUR1, a gene responsible for inositol phosphorylceramide (IPC) synthase activity. Mol Gen Genet, 1999. 261(2): p. 290-6.
34. Zhong, W., M. W. Jeffries, and N. H. Georgopapadakou, Inhibition of inositol phosphorylceramide synthase by aureobasidin A in Candida and Aspergillus species. Antimicrob Agents Chemother, 2000. 44(3): p. 651-3.
35. Robinson, K. A. and S. M. Beverley, Improvements in transfection efficiency and tests of RNA interference (RNAi) approaches in the protozoan parasite Leishmania. Mol Biochem Parasitol, 2003. 128(2): p. 217-28.
36. Kakeya, H., et al., Genetic analysis of azole resistance in the Darlington strain of Candida albicans. Antimicrob Agents Chemother, 2000. 44(11): p. 2985-90.
37. Diaz-Guerra, T. M., et al., A point mutation in the 14alpha-sterol demethylase gene cyp51A contributes to itraconazole resistance in Aspergillus fumigatus. Antimicrob Agents Chemother, 2003. 47(3): p. 1120-4.
38. Sanglard, D., et al., Amino acid substitutions in the cytochrome P-450 lanosterol 14alpha-demethylase (CYP51A1) from azole-resistant Candida albicans clinical isolates contribute to resistance to azole antifungal agents. Antimicrob Agents Chemother, 1998. 42(2): p. 241-53.
39. Mouritsen, O. G. and M. J. Zuckermann, What's so special about cholesterol? Lipids, 2004. 39(11): p. 1101-13.
40. Brown, D. A. and E. London, Functions of lipid rafts in biological membranes. Annu Rev Cell Dev Biol, 1998. 14: p. 111-36.
41. Schuck, S., et al., Resistance of cell membranes to different detergents. Proc Natl Acad Sci USA, 2003. 100(10): p. 5795-800.
42. Proszynski, T. J., et al., Plasma membrane polarization during mating in yeast cells. J. Cell Biol. 2006 Jun. 19; 173(6):861-6.
43. Grossmann, G., et al., Lipid Raft-Based Membrane Compartmentation of a Plant Transport Protein Expressed in Saccharomyces cerevisiae. Eukaryot Cell, 2006. 5(6): p. 945-53.
44. Buchanan, K. T., et al., A flagellum-specific calcium sensor. J Biol Chem, 2005. 280(48): p. 40104-11.
45. Godsel, L. M. and D. M. Engman, Flagellar protein localization mediated by a calcium-myristoyl/palmitoyl switch mechanism. Embo J, 1999. 18(8): p. 2057-65.
46. Denny, P. W., et al., Acylation-dependent protein export in Leishmania. J Biol Chem, 2000. 275(15): p. 11017-25.
47. Yoshida, Y., CytochromeP-450 of fungi: primary target for azole antifungals. Current Topics in Medical Mycology (McGinnis, M. R. Eds), 1988. 2: p. 398-418.
48. Vanden Bossche, H., G. Willemsens, and P. Marichal, Anti-Candida drugs—the biochemical basis for their activity. Crit. Rev Microbiol, 1987. 15(1): p. 57-72.
49. Lira, R., et al., Mechanism of action of anti-proliferative lysophospholipid analogues against the protozoan parasite Trypanosoma cruzi: potentiation of in vitro activity by the sterol biosynthesis inhibitor ketoconazole. J Antimicrob Chemother, 2001. 47(5): p. 537-46.
50. Pike, L. J., Rafts defined: a report on the Keystone Symposium on Lipid Rafts and Cell Function. J Lipid Res. 2006 July; 47(7):1597-8.
51. Zhang, K., et al., Leishmania salvage and remodelling of host sphingolipids in amastigote survival and acidocalcisome biogenesis. Mol Microbiol, 2005. 55(5): p. 1566-78.
52. Kapler, G. M., C. M. Coburn, and S. M. Beverley, Stable transfection of the human parasite Leishmania major delineates a 30-kilobase region sufficient for extrachromosomal replication and expression. Mol Cell Biol, 1990. 10(3): p. 1084-94.
53. Zhang, K., Barron, T., Turco, S. J., and Beverley, S. M., The LPG1 gene family of Leishmania major. Mol. Biochem. Parasitol., 2004. 136: p. 11-23.
54. Folch, J., M. Lees, and G. H. Sloane Stanley, A simple method for the isolation and purification of total lipides from animal tissues. J Biol Chem, 1957. 226(1): p. 497-509.

What is claimed is:

1. A composition for inhibiting protozoan growth comprising a first lipid synthesis inhibitor and a second lipid synthesis inhibitor, wherein
a. the ratio of the EC50 of the first lipid synthesis inhibitor alone to the EC50 of the composition is at least ten, and
b. the ratio of the EC50 of the second lipid synthesis inhibitor alone to the EC50 of the composition is at least ten.

2. The composition of claim 1, wherein
a. the ratio of the EC50 of the first lipid synthesis inhibitor alone to the EC50 of the composition is at least twenty, and
b. the ratio of the EC50 of the second lipid synthesis inhibitor alone to the EC50 of the composition, is at least twenty.

3. The composition of claim 2, wherein
a. the ratio of the EC50 of the first lipid synthesis inhibitor alone to the EC50 of the composition is at least fifty, and
b. the ratio of the EC50 of the second lipid synthesis inhibitor alone to the EC50 of the composition, is at least fifty.

4. The composition of claim 3, wherein
a. the ratio of the EC50 of the first lipid synthesis inhibitor alone to the EC50 of the composition is at least one hundred, and
b. the ratio of the EC50 of the second lipid synthesis inhibitor alone to the EC50 of the composition, is at least one hundred.

5. The composition of claim 1, wherein the first lipid synthesis inhibitor is selected from the group consisting of an ergosterol synthesis inhibitor, a sphingolipid synthesis inhibitor, and an ether phospholipid synthesis inhibitor.

6. The composition of claim 5, wherein the ergosterol synthesis inhibitor is a lanosterol 14α-demethylase inhibitor.

7. The composition of claim 6, wherein the lanosterol 14α-demethylase inhibitor is itraconazole or ketaconazole.

8. The composition of claim 5, wherein the sphingolipid synthesis inhibitor is selected from the group consisting of a serine palmitoyltransferase inhibitor, an inositol phosphorlyceramide synthase inhibitor, and a sphingolipid salvage inhibitor.

9. The composition of claim 5, wherein the first lipid synthesis inhibitor is an ether phospholipid synthesis inhibitor.

10. The composition of claim 9, wherein the ether phospholipid synthesis inhibitor is an alkyl-dihydroxyacetonephosphate transferase inhibitor.

11. The composition of claim 5, wherein the first lipid synthesis inhibitor is a sphingolipid synthesis inhibitor.

12. The composition of claim 1, wherein the second lipid synthesis inhibitor is selected from a different class of lipid synthesis inhibitors than the first lipid synthesis inhibitor.

13. The composition of claim 1, wherein the first lipid synthesis inhibitor is an ergosterol synthesis inhibitor and the second lipid synthesis inhibitor is a sphingolipid synthesis inhibitor.

14. The composition of claim 1, wherein the first lipid synthesis inhibitor is an ergosterol synthesis inhibitor and the second lipid synthesis inhibitor is an ether phospholipid synthesis inhibitor.

15. The composition of claim 1, wherein the first lipid synthesis inhibitor is a sphingolipid synthesis inhibitor and the second lipid synthesis inhibitor is an ether phospholipid synthesis inhibitor.

16. The composition of claim 13, wherein the first lipid synthesis inhibitor is itraconazole or ketaconazole.

17. The composition of claim 14, wherein the first lipid synthesis inhibitor is itraconazole or ketaconazole.

18. A method for inhibiting protozoan growth, the method comprising contacting the protozoan with an effective amount of a composition comprising a first lipid synthesis inhibitor and a second lipid synthesis inhibitor, wherein
a. the ratio of the EC50 of the first lipid synthesis inhibitor alone to the EC50 of the composition is at least ten, and
b. the ratio of the EC50 of the second lipid synthesis inhibitor alone to the EC50 of the composition is at least ten.

19. The method of claim 18, wherein
a. the ratio of the EC50 of the first lipid synthesis inhibitor alone to the EC50 of the composition is at least twenty, and
b. the ratio of the EC50 of the second lipid synthesis inhibitor alone to the EC50 of the composition is at least twenty.

20. The method of claim 19, wherein
a. the ratio of the EC50 of the first lipid synthesis inhibitor alone to the EC50 of the composition is at least fifty, and
b. the ratio of the EC50 of the second lipid synthesis inhibitor alone to the EC50 of the composition is at least fifty.

21. The method of claim 20, wherein
a. the ratio of the EC50 of the first lipid synthesis inhibitor alone to the EC50 of the composition is at least one hundred, and
b. the ratio of the EC50 of the second lipid synthesis inhibitor alone to the EC50 of the composition is at least one hundred.

22. The method of claim 18, wherein the protozoan is selected from the group consisting of Giardia, Trichomonas, Leishmania, Trypansosoma, Entamoeba, Plasmodium, Cryptosporidium, Toxoplasma, Sarcocystis, Theileria, Babesia, and Eimeria.

23. The method of claim 22, wherein the protozoan is Leishmania.

24. The method of claim 18, wherein the first lipid synthesis inhibitor is selected from the group consisting of an ergosterol synthesis inhibitor, a sphingolipid synthesis inhibitor, and an ether phospholipid synthesis inhibitor.

25. The method of claim 24, wherein the ergosterol synthesis inhibitor is a lanosterol 14α-demethylase inhibitor.

26. The method of claim 25, wherein the lanosterol 14α-demethylase inhibitor is itraconazole or ketaconazole.

27. The method of claim 24, wherein the sphingolipid synthesis inhibitor is selected from the group consisting of a serine palmitoyltransferase inhibitor, an inositol phosphorlyceramide synthase inhibitor, and a sphingolipid salvage inhibitor.

28. The method of claim 24, wherein the ether phospholipid inhibitor is an alkyl-dihydroxyacetonephosphate transferase inhibitor.

29. The method of claim 18, wherein the second lipid synthesis inhibitor is selected from a different class of lipid synthesis inhibitors than the first lipid synthesis inhibitor.

30. The method of claim 29, wherein the first lipid synthesis inhibitor is an ergosterol synthesis inhibitor and the second lipid synthesis inhibitor is a sphingolipid synthesis inhibitor.

31. The method of claim 29, wherein the first lipid synthesis inhibitor is an ergosterol synthesis inhibitor and the second lipid synthesis inhibitor is an ether phospholipid synthesis inhibitor.

32. The method of claim 29, wherein the first lipid synthesis inhibitor is a sphingolipid synthesis inhibitor and the second lipid synthesis inhibitor is an ether phospholipid synthesis inhibitor.

33. The method of claim 30, wherein the ergosterol synthesis inhibitor is itraconazole or ketaconazole.

34. The method of claim 31, wherein the ergosterol synthesis inhibitor is itraconazole or ketaconazole.

35. A method for treating infection by a protozoan in a subject comprising administering to the subject an effective amount of a pharmaceutical composition comprising a first lipid synthesis inhibitor and a second lipid synthesis inhibitor, wherein
a. the ratio of the EC50 of the first lipid synthesis inhibitor alone to the EC50 of the composition is at least ten, and
b. the ratio of the EC50 of the second lipid synthesis inhibitor alone to the EC50 of the composition is at least ten.

36. The method of claim 35, wherein
a. the ratio of the EC50 of the first lipid synthesis inhibitor alone to the EC50 of the composition is at least twenty, and
b. the ratio of the EC50 of the second lipid synthesis inhibitor alone to the EC50 of the composition is at least twenty.

37. The method of claim 36, wherein
a. the ratio of the EC50 of the first lipid synthesis inhibitor alone to the EC50 of the composition is at least fifty, and
b. the ratio of the EC50 of the second lipid synthesis inhibitor alone to the EC50 of the composition is at least fifty.

38. The method of claim 37, wherein
a. the ratio of the EC50 of the first lipid synthesis inhibitor alone to the EC50 of the composition is at least one hundred, and
b. the ratio of the EC50 of the second lipid synthesis inhibitor alone to the EC50 of the composition is at least one hundred.

39. The method of claim 35, wherein the protozoan is selected from the group consisting of Giardia, Trichomonas, Leishmania, Trypansosoma, Entamoeba, Plasmodium, Cryptosporidium, Toxoplasma, Sarcocystis, Theileria, Babesia, and Eimeria.

40. The method of claim 39, wherein the protozoan is Leishmania.

41. The method of claim 35, wherein the first lipid synthesis inhibitor is selected from the group consisting of an ergosterol synthesis inhibitor, a sphingolipid synthesis inhibitor, and an ether phospholipid synthesis inhibitor.

42. The method of claim 41, wherein the ergosterol synthesis inhibitor is a lanosterol 14α-demethylase inhibitor.

43. The method of claim 42, wherein the lanosterol 14α-demethylase inhibitor is itraconazole or ketaconazole.

44. The method of claim 41, wherein the sphingolipid synthesis inhibitor is selected from the group consisting of a serine palmitoyltransferase inhibitor, an inositol phosphorlyceramide synthase inhibitor, and a sphingolipid salvage inhibitor.

45. The method of claim 41, wherein the first lipid synthesis inhibitor is an ether phospholipid synthesis inhibitor.

46. The method of claim 45, wherein the ether phospholipid synthesis inhibitor is an alkyl-dihydroxyacetonephosphate transferase inhibitor.

47. The method of claim 35, wherein the second lipid synthesis inhibitor is selected from a different class of lipid synthesis inhibitors than the first lipid synthesis inhibitor.

48. The method of claim 47, wherein the first lipid synthesis inhibitor is an ergosterol synthesis inhibitor and the second lipid synthesis inhibitor is a sphingolipid synthesis inhibitor.

49. The method of claim 47, wherein the first lipid synthesis inhibitor is an ergosterol synthesis inhibitor and the second lipid synthesis inhibitor is an ether phospholipid synthesis inhibitor.

50. The method of claim 47, wherein the first lipid synthesis inhibitor is a sphingolipid synthesis inhibitor and the second lipid synthesis inhibitor is an ether phospholipid synthesis inhibitor.

51. The method of claim 48, wherein the first lipid synthesis inhibitor is itraconazole or ketaconazole.

52. The method of claim 49, wherein the first lipid synthesis inhibitor is itraconazole or ketaconazole.


SEQUENCE LISTING (12 sequences)

SEQ ID NO: 1 - protein, 479 aa, Leishmania major
MIGEFFLLLTAGLALYGWYFCKSFNTTRPTDPPVVHGAMPFVGHIIQFGKDPLDFMLNAKKKYGGVFTMNICGNRVTVVGDVHQHNKFFTPRNEILSPREVYSFMVPVFGEGVAYAAPYPRMREQLNFLAEELTVAKFQNFAPSIQHEVRKFMKANWNKDEGEINILDDCSAMIINTACQCLFGEDLRKRLDARQFAQLLAKMESCLIPAAVFLPWILKLPLPQSYRCRDARAELQDILSEIIIAREKEEAQKDSNTSDLLASLLGAVYRDGTRMSQHEVCGMIVAAMFAGQHTSTITTTWSLLHLMDPRNKRHLAKLHQEIDEFPAQLNYDNVMEEMPFAEQCARESIRRDPPLIMLMRKVLKPVQVGKCVVPEGDIIACSPLLSHQDEEAFPNPREWNPERNMKLVDGAFCGFGAGVHKCIGEKFGLLQVKTVLATVLRDYDFELLGPLPEPNYHTMVVGPTASQCRVKYIRKKAAA

SEQ ID NO: 2 - protein, 503 aa, Homo sapiens [sequence omitted]

SEQ ID NO: 3 - protein, 515 aa, Aspergillus fumigatus
MVPMLWLTAYMAVAVLTAILLNVVYQLFFRLWNRTEPPMVFHWVPYLGSTISYGIDPYKFFFACREKYGDIFTFILLGQKTTVYLGVQGNEFILNGKLKDVNAEEVYSPLTTPVFGSDVVYDCPNSKLMEQKKFIKYGLTQSALESHVPLIEKEVLDYLRDSPNFQGSSGRVDISAAMAEITIFTAARALQGQEVRSKLTAEFADLYHDLDKGFTPINFMLPWAPLPHNKKRDAAHARMRSIYVDIITQRRLDGEKDSQKSDMIWNLMNCTYKNGQQVPDKEIAHMMITLLMAGQHSSSSISAWIMLRLASQPKVLEELYQEQLANLGPAGPDGSLPPLQYKDLDKLPFHQHVIRETLRIHSSIHSIMRKVKSPLPVPGTPYMIPPGRVLLASPGVTALSDEHFPNAGCWDPHRWENQATKEQENDKVVDYGYGAVSKGTSSPYLPFGAGRHRCIGEKFAYVNLGVILATIVRHLRLFNVDGKKGVPETDYSSLFSGPMKPSIIGWEKRSKNTSK

SEQ ID NO: 4 - protein, 528 aa, Candida albicans
MAIVETVIDGINYFLSLSVTQQISILLGVPFVYNLVWQYLYSLRKDRAPLVFYWIPWFGSAASYGQQPYEFFESCRQKYGDVFSFMLLGKIMTVYLGPKGHEFVFNAKLSDVSAEEAYKHLTTPVFGTGVIYDCPNSRLMEQKKFAKFALTTDSFKRYVPKIREEILNYFVTDESFKLKEKTHGVANVMKTQPEITIFTASRSLFGDEMRRIFDRSFAQLYSDLDKGFTPINFVFPNLPLPHYWRRDAAQKKISATYMKEIKSRRERGDIDPNRDLIDSLLIHSTYKDGVKMTDQEIANLLIGILMGGQHTSASTSAWFLLHLGEKPHLQDVIYQEVVELLKEKGGDLNDLTYEDLQKLPSVNNTIKETLRMHMPLHSIFRKVTNPLRIPETNYIVPKGHYVLVSPGYAHTSERYFDNPEDFDPTRWDTAAAKANSVSFNSSDEVDYGFGKVSKGVSSPYLPFGGGRHRCIGEQFAYVQLGTILTTFVYNLRWTIDGYKVPDPDYSSMVVLPTEPAEIIWEKRETCMF

SEQ ID NO: 5 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 6 - DNA, 37 bp, Leishmania
tactagagatctccaccatgatcggcgagttcttcct

SEQ ID NO: 7 - DNA, 32 bp, Leishmania
tactagagatctctaagcagccgccttcttcc

SEQ ID NO: 8 - DNA, 36 bp, Leishmania
agggcaagatctccaccatgggtgcttgtgggtcga

SEQ ID NO: 9 - DNA, 60 bp, Leishmania
gtggagatctttacgcgtagtccgggacgtcgtacgggtagcccgcgctctccggcacgt

SEQ ID NO: 10 - DNA, 30 bp, Artificial
  Primer to introduce G49R mutation
acatcatccagttccgcaaggatccgctgg

SEQ ID NO: 11 - DNA, 30 bp, Artificial
  Primer to introduce Y115H mutation
gagggcgtcgcccacgccgcgccatacccg

SEQ ID NO: 12 - DNA, 29 bp, Artificial
  Primer to introduce S382F mutation
catcatcgcctgcttcccgctcctctcgc